(19) United States
(12) Patent Application Publication
(10) Pub. No.: US 2006/0035889 A1
(43) Pub. Date: Feb. 16, 2006

(54) Treatment for methamphetamine addiction and reduction of methamphetamine use using serotonin antagonists

(76) Inventors: Clark E. Tedford, Poulsbo, WA (US); Tavye Celeste Napier, Maywood, IL (US)

Correspondence Address: Marcia S. Kelbon, Esq.;OMEROS CORPORATION, Suite 2600, 1420 Fifth Avenue, Seattle, WA, 98101, US

(21) Appl. No.: 11/205,769

(22) Filed: Aug. 15, 2005

Related U.S. Application Data
(60) Provisional application No. 60/601,690, filed on Aug. 13, 2004.

(51) Int. Cl. A61K 31/551

(52) U.S. Cl. 514/220

(57) ABSTRACT

Methods for screening specific biological endpoints that can be utilized to identify potential therapeutic agents for METH addiction. In one aspect of the invention, the methods involve reversal of behavioral sensitization and/or conditioned place preference in an animal previously treated with METH in the presence of a known amount of a 5-HT_{2A/2C} antagonist or a selective 5-HT_{2C} antagonist, and reversal of the electrophysiological endpoints in a METH-treated animal in the presence of a known amount of the 5-HT_{2A/2C} antagonist or the selective 5-HT_{2C} antagonist. Therapeutic treatment methods for reversing the set of biological endpoints that change in the METH drug addict using mirtazapine, SDZ SER 082, and related serotonin antagonists are also provided. The methods of the invention may be utilized in the identification of potential new therapies for multiple drugs of abuse.

### CROSS-REFERENCE TO RELATED APPLICATION

The present application claims the benefit of the filing date of U.S. Provisional Patent Application No. 60/601,690 filed Aug. 13, 2004.

### GOVERNMENT RIGHTS

The U.S. Government may have certain rights in the invention due to financial support from the following grants: U.S. Public Health Service, National Institute on Drug Abuse grant numbers 016496 and 015760.

### FIELD OF THE INVENTION

The present invention relates to pharmaceutical compositions and methods of treatment for methamphetamine addiction or the prevention of relapsing back to drug taking in the drug-withdrawn patient experiencing or susceptible to same, by administering to the patient an effective amount of mirtazapine, SDZ SER 082 and related 5-HT_{2A/2C} and 5-HT_{2C} subtype receptor antagonists.

### BACKGROUND OF THE INVENTION

Presently, there is no cure for drug addiction. Indeed, the overwhelming majority (up to 85%) of patients undergoing modern day drug rehabilitation relapse back into compulsive drug taking. This relapse is motivated by the intense craving for the drug that is experienced by the drug-withdrawn addict even after years of being drug free. Psychosocial therapy is widely employed for the long-term treatment of drug addiction, but there remains an exceptionally high incidence of relapse to drug taking in the drug-withdrawn addict. Methamphetamine (“METH”) is an increasingly popular psychostimulant/hallucinogenic drug with an extremely high abuse liability. METH (‘meth’, ‘speed’, ‘ice’, ‘crystal’, and ‘crank’) is a Schedule II stimulant that “on the street” comes in forms amenable to smoking, snorting, oral ingestion or injection. METH often is abused in a relatively drawn out “binge and crash” pattern known as a “run”. This typically lasts for several days, during which time the user foregoes food and sleep. Long-term, heavy use can initiate violent rages, induce anxiety, confusion and insomnia, and evoke a number of psychotic features, including intense paranoia, hallucinations, and delusions that endure for years after drug use has ceased. When METH use is stopped, the user experiences a particularly intense craving for the drug that is protracted. There currently is no approved medication or efficacious pharmacotherapy for METH abuse. The present invention relates to pharmaceutical compositions and methods of identifying new pharmacotherapies for METH addiction or the prevention of relapsing back to drug-taking in the drug-withdrawn addict.

Withdrawal from repeated, intermittent administration of psychomotor stimulants like METH is associated with an enduring enhancement in several measures of behavioral function (termed behavioral sensitization). This occurs in all mammals tested (e.g., mice, rats, monkeys and humans). In non-human mammals, the neural changes (neural sensitization) associated with such sensitized behaviors are thought to emulate those that characterize addiction in humans.

With repeated exposure, METH, and all other abused drugs, take on greater and greater significance in humans and in non-human animals. This “enhanced salience” is thought to contribute to drug-craving, and thus underlies the compulsive drug-seeking and eventual relapse to drug-taking that occurs in the drug-withdrawn addict. An important aspect of this phenomenon is attributable to learning to make an association between the rewarding effects of METH and people, places or things that are affiliated with the drug-taking (e.g., a friend, a neighborhood bar or drug paraphernalia). In all species tested (including humans) these drug-associated “cues” also take on enhanced significance with repeated drug exposure, and they then serve as powerful triggers to initiate craving, seeking and relapse in the drug-withdrawn addict. (For example, the effect that seeing a cigarette machine has on an ex-smoker.) It is becoming clear that suitable pharmacotherapy that will keep the addict drug-free will act on targets that can reduce the significance of the drug and its associated cues.

Drug-induced associative learning involves a form of neuronal sensitization. While learning-induced sensitization may share some aspects of the molecular, receptor, and anatomical substrates that are engaged by motor sensitization, it is becoming increasingly clear that these two models of addiction may shed unique insights. Thus, it is advantageous to consider both types of models when assessing the therapeutic potential of novel pharmacologic targets.

A third feature of the addiction phenomenon is the persistence of the brain and behavioral changes that are instigated by repeated exposure to drugs of abuse like METH. This is modeled in non-human animals. In rats, repeated intermittent treatments of moderately low doses (1-3 mg/kg/day) of METH consistently induces sensitized behavioral responding to an acute METH challenge given 5 to 14 days later.[1-6] Depending upon the dose used and the duration of the repeated treatment regimen, sensitized motor response to an acute challenge [5] and expression of place preference [7] occurs months after the last repeated injection.

As stated previously, there are no drugs that have been shown to be effective in preventing relapse in humans. Similarly, at present there are no drugs that are known to reverse METH-induced behavioral or neural sensitization in animal models of human addiction. A role for serotonin (5-hydroxytryptamine; 5-HT) in drug addiction in general and for serotonin receptor antagonists as useful medications in the treatment of METH addiction in particular is not recognized by the limited animal studies in the field. Most studies have evaluated other psychostimulants, i.e., amphetamine and cocaine, or used self-administration paradigms. [8] One study demonstrated that depletion of brain 5-HT (with p-chlorophenylalanine) decreases cocaine-seeking behavior in rats. [9] Similarly, in human cocaine addicts, the craving normally elicited by environmental stimuli previously associated with cocaine administration is decreased following a reduction in brain 5-HT levels by lowering plasma levels of its precursor, tryptophan. [10] The role of various 5-HT receptor subtypes in maintaining METH addiction has not been established.

U.S. Pat. No. 5,039,680 and U.S. Pat. No. 5,198,459 claim the use of 5-HT₃ subtype antagonists in the manufacture of a medicament suitable for the prevention or reduction of dependence on a dependence-inducing agent. Their teachings describe that other dependency-inducing agents (brought on by low parenteral doses, e.g., ranging from about 1 to about 5 mg/kg s.c. in the case of morphine, 0.6 mg/kg s.c. in the case of nicotine, and about 5 mg/kg i.p. in the case of ethanol) commonly act by increasing the release and utilization of the neurotransmitter, dopamine, in brain regions known to be involved in drug addiction (e.g., the nucleus accumbens). Behavioral indices of drug effects, (e.g., stereotypies in the case of morphine, locomotion in the case of nicotine and hypnosis in the case of ethanol), correlate in time with the stimulation of dopamine release. Their work did not examine METH nor address drug addiction effects on serotonergic systems.

It is known that the mechanism of action of METH and related stimulants (i.e., amphetamines) differs from that of morphine, nicotine and ethanol and this contributes to the greater potential for METH to engage brain serotonergic systems. In addition, U.S. Pat. No. 5,039,680 and U.S. Pat. No. 5,198,459 teach that the preferable compounds of the invention are selective 5-HT₃ antagonists that do not significantly block 5-HT₁ or 5-HT₂ receptors. When given in the acute withdrawal period from dosing regimens of cocaine that produce behavioral sensitization, ondansetron, a 5-HT₃ selective antagonist, [11]as well as ketanserin and mianserin [12] reverse the established behavioral sensitization. However, the underlying neuronal sensitization, e.g., neuronal markers for the 5-HT_{2a/2c} receptor function, were not investigated in any of these studies, nor was METH evaluated.

Evaluations of potential addiction therapy on human psychomotor stimulant abusers have focused on antidepressants that are norepinephrine and serotonin selective reuptake inhibitors (SSRIs). However, in controlled clinical trials of METH addicts, imipramine was not found to significantly reduce craving or change the percent of urine samples positive for the stimulant. [13; 14] While several studies have recognized that serotonin plays a role in addiction, [15-17], there is a need for efficacious treatment for METH addiction or relapse prevention for the METH-withdrawal addict.

### SUMMARY OF THE INVENTION

The present invention relates to the ability of certain 5-HT_{2A/2C} receptor antagonists, specifically mirtazapine, as well as selective 5-HT_{2C} receptor antagonists, such as SDZ SER 082 (4,5,7a,8,9,10,11,11a,-octahydro-7H-10-methylindolo[1,7,bc] [2,6]-napthyridine), to nullify or reverse long-lasting neuronal and behavioral sensitization produced by METH. The current invention recognizes that repeated METH exposure modifies the biochemical function and the behavioral effects of 5-HT_{2A/2C} receptors, that these changes persist long after METH is withdrawal, and that post-sensitization pharmacotherapy with certain antagonists to the 5-HT_{2A/2C} subtypes, e.g., mirtazapine, or selective 5-HT_{2C} receptor antagonists, e.g., SDZ SER 082, can reverse neuronal and behavioral sensitization to METH.

A first aspect of the present invention provides methods for the treatment of a mammal suffering from addiction to METH or to another drug by treating the mammal with a therapeutically effective amount of 5-HT_{2A/2C} receptor antagonist or a selective 5-HT₂C receptor antagonist, and compositions including such 5-HT_{2A/2C} receptor antagonists and selective 5-HT_{2C} receptor antagonists, wherein the 5-HT_{2A/2C} receptor antagonist or the selective 5-HT_{2C} receptor antagonist has been screened to determine that it does not potentiate the effect of the drug. Suitably, the methods and compositions of the present invention utilize either a 5-HT_{2A/2C} receptor antagonist with high-affinity for 5-HT_{2C} receptors, or a selective 5-HT_{2C} receptor antagonist.

The present invention provides a method for the treatment of an animal, for example, a mammal including a human patient, suffering from METH addiction, comprising administering an effective amount of mirtazapine. The present invention also provides a method for the treatment of an animal, for example, a mammal including a human patient, suffering from METH addiction, comprising administering an effective amount of SDZ SER 082. The invention also involves the use of mirtazapine or SDZ SER 082 for the manufacture of a medicament for the treatment of METH addiction.

In a first preferred embodiment of the invention, a composition comprising a therapeutically effective amount of mirtazapine or SDZ SER 082 in a pharmaceutically acceptable carrier is administered to a subject suffering from METH addiction, for treating such addiction or for preventing relapse in such a subject.

Without wishing to be bound by theory, the applicant, with the hindsight of the unexpected effect of the invention, believes that the particular pharmacological profile of mirtazapine or SDZ SER 082 is responsible for the efficacy against METH addiction or relapse during withdrawal from METH use.

In a further embodiment of the invention, a composition comprising a therapeutically effective amount of a related 5-HT_{2A/2C} subtype receptor antagonist having a pharmacologic profile similar to mirtazapine in a pharmaceutically acceptable carrier is administered to a subject suffering from METH addiction, for treating such addiction or for preventing relapse in such a subject.

In a further embodiment of the invention, a composition comprising a therapeutically effective amount of a related 5-HT₂C subtype receptor antagonist having a pharmacologic profile similar to SDZ SER 082 in a pharmaceutically acceptable carrier is administered to a subject suffering from METH addiction, for treating such addiction or for preventing relapse in such a subject.

In a still further embodiment of the invention, compositions comprising a therapeutically effective amount of mirtazapine or a related 5-HT_{2A/2C} subtype receptor antagonist having a pharmacologic profile similar to mirtazapine is administered to a patient suffering from addiction to a drug such as methamphetamine, amphetamine, methylenedioxymethamphetamine (MDMA or ecstasy), and other substituted amphetamines, cocaine, alcohol (ethanol), opiates, and nicotine and other substituted amphetamines.

In a still further embodiment of the invention, compositions comprising a therapeutically effective amount of SDZ SER 082 or a related 5-HT_{2C} subtype receptor antagonist having a pharmacologic profile similar to SDZ SER 082 is administered to a patient suffering from addiction to a drug such as methamphetamine, amphetamine, methylenedioxymethamphetamine (MDMA or ecstasy), and other substituted amphetamines, cocaine, alcohol (ethanol), opiates, and nicotine and other substituted amphetamines.

In a further aspect of the invention, screening methods are provided for identifying compounds for the treatment of METH addiction. A first embodiment of the screening method comprises (a) the reversal of behavioral sensitization and/or conditioned place preference (“CPP”) in a METH-treated animal in the presence of a known amount of a compound; and (b) the reversal of the electrophysiological endpoints in a METH-treated animal in the presence of a known amount of the compound. In a preferred aspect of the invention, the compound is a 5-HT antagonist, and in a more preferred aspect the compound is a 5-HT_{2A/2C} or 5-HT_{2C} antagonist.

An alternate embodiment of the screening method comprises (a) the reversal of behavioral sensitization and/or conditioned place preference in a METH-treated animal in the presence of a known amount of a compound; and (b) the modification of biochemical endpoints in a METH-treated animal in the presence of a known amount of the compound, such as the reversal of behavioral sensitization comprises an attenuation of up-regulated 5-HT_{2A/2C} receptor function in the brain and an attenuation in METH-induced changes in gene transcriptional modulators such as cAMP-response element binding protein. In a preferred aspect of the invention, the compound is a 5-HT antagonist, and in a more preferred embodiment the compound is a 5-HT_{2A/2C} or 5-HT_{2C} antagonist.

The screening methods of the present invention may also be used to identify compounds for the treatment of addiction to other drugs, including an addictive condition involving one or more of the following drugs: methamphetamine, amphetamine, methylenedioxymethamphetamine (MDMA or ecstasy), and other substituted amphetamines, cocaine, alcohol (ethanol), opiates, and nicotine and other substituted amphetamines.

### DESCRIPTION OF THE DRAWINGS

The present invention may be better understood in view of the accompanying drawings, wherein:

FIGS. 1a and 1b demonstrate that repeated METH treatment induces behavioral sensitization with a 3-day challenge of METH (1 mg/kg).

FIGS. 2a and 2b demonstrate that repeated METH treatment induces behavioral sensitization with a 3-day challenge of METH (1 mg/kg).

FIGS. 3a and 3b illustrate the effect of ketanserin treatment (1 mg/kg) on METH-induced behavioral sensitization.

FIGS. 4a and 4b illustrate the effect of mianserin treatment (2.5 mg/kg) on METH-induced behavioral sensitization.

FIGS. 5a and 5b illustrate the effect of mianserin treatment (1 mg/kg) on METH-induced behavioral sensitization.

FIGS. 6a and 6b illustrate the effect of mirtazapine treatment (5 mg/kg) on METH-induced behavioral sensitization.

FIG. 7 illustrates a METH-induced conditioned place preference (CPP) dose-response study. CPP expression 48 hours following a single-pairing of (A) 0 mg/kg (i.e., saline alone), (B) 0.1 mg/kg, (C) 0.3 mg/kg or (D) 1.0 mg/kg METH is shown. Data (collected on Day 3) were analyzed using a paired t-test for within group comparisons (*p<0.05, n/s=not significant, n=8/group).

FIG. 8 illustrates CPP expression following (A) vehicle treatment during METH-withdrawal or (B) mirtazapine treatment during METH-withdrawal. Animals received 3 pairings with 1 mg/kg ip METH on alternate days. Ten once-daily injections of 5 mg/kg ip mirtazapine or its vehicle were given during the withdrawal phase. Data (collected on day 20) were analyzed using a paired t-test for within group comparisons (*p<0.05, n/s=not significant, n=8/group).

FIG. 9 illustrates motor activity in response to 0.1 mg/kg METH challenge on day 4. Activities shown are (A) horizontal activity, (B) vertical activity, (C) stereotypy count, (D) number of rears, (E) rearing time and (F) distance traveled. Animals had previously received a single injection of 0, 0.1, 0.3 or 1.0 mg/kg METH on day 1. Data (collected on Day 4) were analyzed using ANOVA with post-hoc Newman-Keuls (*p<0.05, **p<0.01, ***p<0.001, n=8/group).

FIG. 10 illustrates persistence of METH-induced motor sensitization. Number of rears on conditioning days 1, 3 and 5 and METH challenge of day 22 after (A) vehicle treatment during METH withdrawal or (B) mirtazapine treatment during METH withdrawal. Data were analyzed using a repeated measures ANOVA with post-hoc Newman-Keuls *p<0.05, ***p<0.001, ns=not significant vs. day 1. Numbers in parentheses below the bars indicate day of study.

FIG. 11 illustrates mirtazapine reversal of METH-induced CPP. CPP expression was measured 48 hours following a single-pairing of 1.0 mg/kg METH (i.e., on Day 4), and 24 hours after home cage administration of (A) vehicle, (B) 0.5 mg/kg mirtazapine, (C) 1.0 mg/kg mirtazapine or (D) 5.0 mg/kg mirtazapine. Data were analyzed using a paired t-test for within group comparisons (p*<0.05, n=8/group).

FIG. 12 illustrates CPP expression following a “reinstatement” METH injection, and the ability of mirtazapine to prevent this effect. (A) shows vehicle treatment during METH withdrawal, and (B) shows mirtazapine treatment during METH withdrawal. Data (collected on Day 25) were analyzed using a paired t-test for within group comparisons, *p<0.05, ns=not significant.

FIG. 13 illustrates that the 5-HT_{2C} antagonist, SDZ SER 082, reverses METH-CCP. CCP expression 48 hours following a single-pairing of 1.0 METH is shown. The CPP test was carried out 24 hours after home cage administration of (A) 0 mg/kg, (B) 0.03 mg/kg, (C) 0.1 mg/kg or (D) 1.0 mg/kg SDZ SER 082. Data (collected on Day 4) were analyzed using a paired t-test for within group comparisons (*p<0.05, ns=not significant).

FIG. 14 illustrates that pCREB and the ratio of pCREB to CREB is increased in the frontal cortex, nucleus accumbens and ventral pallidum of methamphetamine sensitized rats after 3 days withdrawal. In the cortex, there was an effect of repeated treatment (p=0.02), withdrawal time (p=0.02) and a treatment-withdrawal time interaction, (p=0.02). Likewise, pCREB/CREB showed an effect of pretreatment (p=0.03), withdrawal time (p=0.003) and pretreatment-withdrawal time interaction (p=0.005). In the nucleus accumbens, mANOVA evaluations showed treatment effects for pCREB (p=0.002) and pCREB/CREB (p=0.0036). For the ventral pallidum, a mANOVA revealed a effect of repeated treatment on pCREB levels (p=0.04), and for the pCREB/CREB ratio (p=0.0018). Asterisks above graphs indicate significance using a mANOVA while asterisks above individual bars indicate significant difference between pretreatment groups using a post-hoc Newman-Keuls, *p<0.05; **p<0.01. Immunoblots above graphs illustrate pCREB or CREB bands of tissue from the same treatment group/withdrawal times as each bar.

FIG. 15 illustrate that ΔFosB is increased in the nucleus accumbens and ventral pallidum of 3 day-withdrawn methamphetamine-sensitized rats; this increase persists to 14 days withdrawal in the ventral pallidum. For the accumbens (left), a mANOVA revealed a treatment effect (p=0.009), for the ventral pallidum (right), there was a treatment effect (p=0.0003). Asterisks above graphs indicate significance using a mANOVA while asterisks above individual bars indicate significant difference between pretreatment groups using a post-hoc Newman-Keuls, *p<0.05; **p<0.01. Representative immunoblots from the different assays are respectively illustrated above each bar.

FIG. 16 illustrates the rate-enhancing effects of an acute challenge of METH or the 5-HT_{2A/2C} agonist DOI on ventral pallidal neuronal firing is enhanced in METH-sensitized rats. Neuronal spiking was obtained in anesthetized rats three days after the last of five once-daily sc injections of 2.5 mg/kg METH or saline. METH (A) or DOI (B) was administered i.v. in a cumulative fashion. Left panels, averaged dose-effect curves. Right panels, bar graphs showing potency (ED₅₀) and efficacy (Emax). Data are mean±SEM; *, t-test, p<0.05. The keys list the chronic treatment. In the METH-sensitized rats, only three neurons were tested with 4 mg/kg iv METH; thus the large SEM.

FIG. 17 illustrates the rate-enhancing effects of an acute challenge of METH on ventral pallidal neuronal firing is diminished in persistently METH-sensitized rats. This contrasts the firing rate enhancement seen at 3 days withdrawal (see previous FIG.). Neuronal spiking was obtained in anesthetized rats 30 days after the last of five once-daily sc injections of 2.5 mg/kg METH or saline. METH was administered i.v. in a cumulative fashion. A) Representative histograms illustrating ventral pallidal neuronal responses to i.v. METH in rats that were pretreated with either saline (upper panel) or METH. B) Left panel, averaged dose-effect curves for ventral pallidal neuronal responses to i.v. METH. All data are mean±SEM; *,p<0.05, **p<0.01 (rmANOVA with post-hoc Newman-Keuls). Lower right panels, bar graphs showing potency (ED₅₀) and efficacy (Emax)*, p<0.05, t-test. The keys list the repeated pretreatment.

### DESCRIPTION OF SPECIFIC EMBODIMENTS

Definitions

The term “serotonin surrogate” refers to a compound that acts as a ligand for a serotonin receptor and modulates the activity of the serotonin receptor in a similar fashion to the natural ligand serotonin.

The term “antagonist” refers to a compound that decreases the strength or duration of the activity mediated by the 5-HT receptor variants.

The present inventors, by evaluating processes that endure long after withdrawal from repeated treatments of METH, have identified a pattern of behavioral, biochemical, genetic and electrophysiological changes that occur in the brain following METH-induced sensitization. Thus, the present invention teaches a reliable set of biological endpoints that can be utilized to identify potential therapeutic agents in METH addiction. In this invention the therapeutic focus is on serotonergic agents that have been discovered to reverse the set of biological endpoints that change in the METH drug addict. More broadly, the methods used in the invention may be utilized in the identification of potential new therapies for multiple drugs of abuse.

The invention further teaches the discovery of 5-HT antagonists that have pharmacologic profiles that are similar to mirtazapine or SDZ SER 082 and that may be useful in the treatment and management of addiction to a variety of substances of abuse including but not limited to METH, methylenedioxymethamphetamine (DMA or ecstasy), amphetamine, cocaine, alcohol (ethanol), opiates, and nicotine. Thus, the battery of tests of this invention can be employed as screening systems to identify other 5-HT antagonists that would be useful in the treatment of drug addiction. These systems provide methods for identifying any appropriate known ligand, which would be therapeutically useful in the treatment of METH addiction or broadly any drug addiction.

Serotonergic antagonists identified by the methods of the present invention are also included in the present invention as are pharmaceutical compositions comprising the identified antagonists and a pharmaceutically acceptable carrier. The present invention, in one aspect, provides compounds, either 5-HT antagonists, identified by the methods disclosed herein, which compounds are useful for the treatment of diseases, disorders and conditions associated with drug addiction. Some such conditions include those mentioned above. Compounds (that is, 5-HT antagonists) identified according to the methods disclosed herein may be used alone at appropriate dosages defined by routine testing in order to obtain optimal modulation (either activation or inhibition) of the battery of tests while minimizing any potential toxicity. In addition, co-administration or sequential administration of other agents may be desirable.

The neurotransmission modulating compositions employed in the practice of the present invention may comprise any of a wide variety of 5-HT antagonists that have pharmacologic profiles similar to mirtazapine or SDZ SER 082. Useful agents include: the compounds disclosed in U.S. Pat. No. 4,062,848 issued Dec. 13, 1977 to Willem Jacob van der Burg for “Tetracyclic Compounds,” the disclosure of which is hereby incorporated herein by reference in its entirety. U.S. Pat. No. 4,062,848 and U.S. Pat. No. 4,025,513 discloses Mirtazapine and related structural analogs, which can be generally described as dibenzo-pyrazino-azepine or benzo-pyrido-pyrazino-azepine derivatives. Further, the present invention may comprise isomers of the above motifs as described in EPA 447, 857 and further described in U.S. Pat. No. 5,407,933 and U.S. Pat. No. 5,476,848. The present invention does not comprise mianserin, which unlike mirtazapine, potentiates the effects of METH at certain doses and thus would not provide a beneficial pharmacological profile.

Other suitable 5-HT antagonists may include ergonovine (Ergotrate), pizotifen, Ondansetron (Zofran), ritanserin, clozapine (Clozaril), risperidone (Risperdal), methysergide (Sansert), and cyproheptadine (Periactin).

Other compounds contemplated by the invention are those disclosed in U.S. Pat. No. 5,198,459 issued Mar. 30, 1993 to Assunta Imperato, et al., including, for example, indol-3-yl-carboxylic acid-endo-8-methyl-8-aza-bicyclo[3,2,1]-oct-3-yl-ester; benzo[b]thiophen-3-yl-carboxylic acid-endo-9-methyl-azabicyclo-[3,3,1]non-3-yl-ester; 5-fluoro-1-methyl-indol-3-yl-carboxylic acid-endo-9-methyl-9-aza-bicyclo[3,3,1]non-3-yl-ester; 1,2,3,9-tetrahydro-9-methyl-3-[(2-methyl-1H-imidazol-1-yl)-methyl-4H-carbazol-4-one; 1-methyl-indazol-3-yl-carboxylic acid-9-methyl-9-aza-bicyclo-[3,3,1]-non-3.alpha.-yl-amide; endo-4-amino-5-chloro-2-methoxy-N-(1-azabicyclo[3,3,1]non-4-yl)-benzamide; and 3-[5-methyl-1H-imidazol-4-yl]-1-(1-methyl-1H-indol-3-yl)-1-propanone.

One class of compounds that may be useful in the treatment of METH addiction in accordance with the present invention include the tetracyclic compounds of U.S. Pat. No. 4,062,848, of the formula: 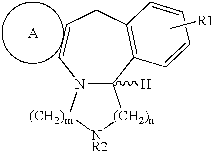
or a salt thereof, wherein
A represents a pyridine ring or a halogen substituted pyridine ring,
R₁ represents hydrogen, C₁-C₆ alkyl, C₁-C₆ alkoxy, C₁-C₆ alkylthio, halogen, OH, SH or CF₃
R₂ represents hydrogen or a lower alkyl or aralkyl group and
n and m may each be 1, 2 or 3 with the proviso that the sum of in and n must be 2, 3 or 4.

Various other 5-HT₂ and 5-HT₃ postsynaptic receptor antagonists may likewise be employed in the treatment of METH addiction in the broad practice of the present invention providing they have similar pharmacologic profiles to mirtazapine.

One presently preferred METH addiction or relapse prevention therapeutic composition in the general practice of the present invention comprises mirtazapine, a piperazinoazepine characterized as (i) a presynaptic α₂ antagonist that acts to increase noradrenergic and serotonergic neurotransmission, and (ii) a postsynaptic serotonergic 5-HT₂ and 5-HT₃ antagonist. Mirtazapine, or 6-azamianserin, includes the compound, 1,2,3,4,10,14b-hexahydro-2-methyl-pyrazino[2,1-a]pyrido[2,3-c]benzazepine) in racemic forms. The S(+) enantiomer has the formula:

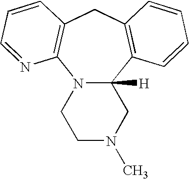

Mirtazapine is sold in racemic mixture under the trademark REMERON (NV Organon, Oss, The Netherlands) as an FDA-approved drug for the treatment of depression, for which indication the usual daily dose is on the order of from about 15 to about 60 milligrams (mg.). Mirtazapine is described in U.S. Pat. No. 5,977,099 as useful for depression only with at least one SSRI. Mirtazapine is also described in U.S. Pat. No. 6,281,207 and U.S. patent application 2002/0035057 as having utility only in specific movement disorders. The present invention contemplates the use of the racemic mixture mirtazapine, as well as the use of substantially pure enantiomeric components thereof, e.g., produced by chiral synthesis or by appropriate racemic separation technique, as well as the use of non-racemic forms of the respective R(−)- and S(+)-racemic forms.

Recognized receptor affinities (Kᵢ in nM) for mirtazapine are as follows: α₁ 500; α₂, 65; 5-HT_{1A}, greater than 1,000; 5-HT_{2A}, 6; 5-HT_{2C}, 12; 5-HT₃, 8; D₁, greater than 1,000; D₂, greater than 1,000; SERT, greater than 1,000; NET, greater than 1,000; H₁, 0.5. [18;19] Serotonin antagonists having similar pharmacologic profiles are within the scope of the present invention.

In addition, SDZ SER 082 (4,5,7a,8,9,10,11,11 a,-octahydro-7H-10-methylindolo[1,7,bc][2,6]-napthyridine, available from Tocris Biosciences with permission of Novartis Pharma AG) has been disclosed as a selective 5-HT_{2C} receptor antagonist. Recognized receptor affinities (Kᵢ in nM) for SDZ SER 082 are as follows: a, greater than 1,000; 5-HT_{1A}, 800; 5-HT_{2A}, 600; 5-HT_{2C}, 15; 5-HT₃, greater than 1,000; D₁, greater than 1,000; D₂, greater than 1,000.[20] Distinct physiological roles have been attributed to either 5-HT_{2A} or 5-HT_{2C} receptors.[21-26] In studies evaluating the role of 5-HT receptor subtypes in cocaine seeking behaviors, rats were trained to press a lever for cocaine (0.5 mg/kg/infusion, iv) paired with the cue (light+tone).[21] After stabilization of self-administration response, the animals underwent daily extinction sessions during which responding had no consequences. The cocaine seeking behavior was reinstated by cocaine priming (10 mg/kg, ip) or by presentation of the cue. Neither SR 46349B (0.25-1 mg/kg) nor SDZ SER 082 (0.25-1 mg/kg) altered the maintenance of cocaine self-administration.[21] SDZ SER 082 failed to alter both cue- and cocaine priming-induced reinstatement.[21] These findings indicated that 5-HT_{2A} and 5-HT_{2C} receptors are not significant to cocaine rewarding effects.[21] However, they show the importance of the 5-HT_{2A} receptors (but not 5-HT_{2C} receptors) in cocaine-priming- and cue-provoked reinstatement.[21] The results of the current invention indicate the usefulness of 5-HT_{2A} receptor antagonists like SDZ SER 082 in reversing the METH sensitization are particularly surprising in view of the above findings.

SDZ SER 082 is a 6,5,6,6 fused tetracyclic compound containing two nitrogens, one at the B-C ring junction (i.e. the indolizidine nitrogen), and the other at the D ring.

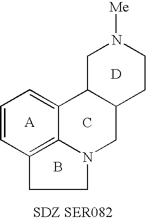

Various other 5-HT_{2A/2C} and 5-HT_{2C} postsynaptic receptor antagonists of this class may likewise be employed in the treatment of METH addiction in the broad practice of the present invention providing they have similar pharmacologic profiles to SDZ SER 082.

More generally, and with reference herein to specific compounds or classes of compounds as usefully employed in the practice of the invention, such compounds or classes of compounds are intended to be broadly construed to encompass within the scope thereof salts, esters, amides, carbamates, solvates, polymorphs, hydrates, affinity reagents, tautomeric forms, optical isomers that are either dextrorotatory or levorotatory, respective dextrorotatory or levorotatory pure preparations, and mixtures thereof, stereoisomers (enantiomers and diastereoisomers) and mixtures thereof, derivatives and/or prodrugs of such compounds, in either crystalline or amorphous form. The esters, amides and carbamates are preferably hydrolyzable and are more preferably biohydrolyzable. The salts are preferably pharmaceutically acceptable salts.

The compounds described herein may also be substituted by substituents that are sterically acceptable, chemically and biochemically compatible and which do not preclude the efficacy of the compound for its intended utility of combating METH addiction. In enantiomeric forms, compounds of the invention include individual enantiomers of the compounds in single species form substantially free of its optical antipode, as well as in admixture (in mixtures of enantiomeric pairs and/or in mixtures of multiple enantiomer species).

Pharmaceutically acceptable esters of compounds of the invention include carboxylic acid esters of hydioxy groups in such compounds in which the non-carbonyl moiety of the carboxylic acid portion of the ester grouping is selected from straight or branched chain alkyl (e.g. n-propyl, t-butyl, n-butyl), alkoxyalkyl (e.g. methoxymethyl), arylalkyl (e.g. benzyl), aryloxyalky (e.g. phenioxymethiyl), and aryl (e.g. phenyl); alkyl-, aryl-, or arylalkylsulfonyl (e.g. methaniesulfonyl); amino acid esters (e.g. L-valyl or L-isoleucyl); dicarboxylic acid esters (e.g. hemisuccinate); carbonate esters (e.g. ethoxycarbonyl); carbamate esters (e.g. dimethylaminocarbonyl, (2-aminoethyl)aminocarbonyl); and inorganic esters (e.g. mono-, di- or triphosphate).

Pharmaceutically acceptable salts of the compounds of the invention and physiologically functional derivatives thereof include salts derived from an appropriate base, such as an alkali metal (for example, sodium, potassium), an alkaline earth metal (for example, calcium, magnesium), ammonium and NX⁴⁺ (wherein X is C₁-C₄ alkyl). Pharmaceutically acceptable salts of an amino group include salts of: organic carboxylic acids Such as acetic, lactic, tartaric, malic, lactobionic, fumaric, and succinic acids; organic sulfonic acids such as methaniesulfollic, ethanesulfonic, isethioniic, benzenlesulfonic and p-toluenesulfoniic acids; and inorganic acids such as hydrochloric, hydrobromic, sulfuric, phosphoric and sulfamic acids. Pharmaceutically acceptable salts of a compound having a hydroxy group consisting of the anion of said compound in combination with a suitable cation such as Na⁺, NX⁴⁺ or NX⁴⁺ (wherein X is for example a C₁-C₄ alkyl group).

For therapeutic use, salts of compounds of the invention will be pharmaceutically acceptable, i.e., they will be salts derived from a pharmaceutically acceptable acid or base. However, salts of acids or bases that are not pharmaceutically acceptable may also find use, for example, in the preparation or purification of a pharmaceutically acceptable compound. All salts, whether or not derived from a pharmaceutically acceptable acid or base, are within the scope of the present invention.

The present invention also provides suitable topical, oral, systemic and parenteral pharmaceutical formulations for use in methods of treatment of diseases and disorders associated drug abuse. The compositions containing compounds identified according to this invention as the active ingredient for use in the modulation of METH addiction can be administered in a wide variety of therapeutic dosage forms in conventional vehicles for administration. For example, the compounds or modulators can be administered in such oral dosage forms as tablets, capsules (each including timed release and sustained release formulations), pills, powders, granules, elixirs, tinctures, solutions, suspensions, syrups and emulsions, or by injection. Likewise, they may also be administered in intravenous (both bolus and infusion), intraperitoneal, subcutaneous, topical with or without occlusion, or intramuscular form, all using forms well known to those of ordinary skill in the pharmaceutical arts. An effective but non-toxic amount of the compound desired can be employed as a human serotonin receptor variant modulating agent.

The daily dosage of the compounds may be varied over a wide range from 0.01 to 1,000 mg per patient, per day. For oral administration, the compositions are preferably provided in the form of scored or unscored tablets containing 0.01, 0.05, 0.1, 0.5, 1.0, 2.5, 5.0, 10.0, 15.0, 30.0, and 50.0 milligrams of the active ingredient for the symptomatic adjustment of the dosage to the patient to be treated. An effective amount of the drug is ordinarily supplied at a dosage level of from about 0.0001 mg/kg to about 100 mg/kg of body weight per day. The range is more particularly from about 0.001 mg/kg to 10 mg/kg of body weight per day. The dosages of the drug are adjusted when combined to achieve desired effects. On the other hand, dosages of these various agents may be independently optimized and combined to achieve a synergistic result wherein the pathology is reduced more than it would be if either agent were used alone.

For example, the mirtazapine composition may be administered to a human patient at a daily dose in the range of from about 10 to about 100 milligrams, and more preferably from about 15 to about 50 milligrams. The SDZ SER 082 composition may be administered to a human patient at a daily dose in the range of from about 1 to about 100 milligrams, and more preferably from about 10 to about 100 milligrams. Such dosage may be administered in a single or multiple dosage form, e.g., an oral tablet or capsule.

Advantageously, compounds of the present invention may be administered in a single daily dose, or the total daily dosage may be administered in divided doses of two, three or four times daily. Furthermore, compounds or modulators for the present invention can be administered in intranasal form via topical use of suitable intranasal vehicles, or via transdermal routes, using those forms of transdermal skin patches well known to those of ordinary skill in that art. To be administered in the form of a transdermal delivery system, the dosage administration will, of course, be continuous rather than intermittent throughout the dosage regimen. For combination treatment with more than one active agent, where the active agents are in separate dosage formulations, the active agents can be administered concurrently, or they each can be administered at separately staggered times.

The dosage regimen utilizing the compounds of the present invention is selected in accordance with a variety of factors including type, species, age, weight, sex and medical condition of the patient; the severity of the condition to be treated; the route of administration; the renal and hepatic function of the patient; and the particular compound thereof employed. A physician or veterinarian of ordinary skill can readily determine and prescribe the effective amount of the drug required to prevent, counter or arrest the progress of the condition. Optimal precision in achieving concentrations of drug within the range that yields efficacy without toxicity requires a regimen based on the kinetics of the drug's availability to target sites. This involves a consideration of the distribution, equilibrium, and elimination of a drug.

In the methods of treatment of the present invention, the compounds herein described in detail can form the active ingredient, and are typically administered in admixture with suitable pharmaceutical diluents, excipients or carriers (collectively referred to herein as “carrier” materials) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include, without limitation, sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

For liquid forms the active drug component can be combined in suitably flavored suspending or dispersing agents such as the synthetic and natural gums, for example, tragacanth, acacia, methyl-cellulose and the like. Other dispersing agents that may be employed include glycerin and the like. For parenteral administration, sterile suspensions and solutions are desired. Isotonic preparations that generally contain suitable preservatives are employed when intravenous administration is desired.

Topical preparations containing the active drug component can be admixed with a variety of carrier materials well known in the art, such as, e.g., alcohols, aloe vera gel, allantoin, glycerine, vitamin A and E oils, mineral oil, PPG2 myristyl propionate, and the like, to form, e.g., alcoholic solutions, topical cleansers, cleansing creams, skin gels, skin lotions, and shampoos in cream or gel formulations.

The compounds of the present invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Compounds of the present invention may also be delivered by the use of monoclonal antibodies as individual carriers to which the compound molecules are coupled. The compounds or modulators of the present invention may also be coupled with soluble polymers as targetable drug carriers. Such polymers can include polyvinyl-pyrrolidone, pyran copolymer, polyhydroxypropylmethacrylamidephenol, polyhydroxy-ethylaspartamidephenol, or polyethyleneoxidepolylysine substituted with palmitoyl residues. Furthermore, the compounds or modulators of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydro-pyrans, polycyanoacrylates and cross-linked or amphipathic block copolymers of hydrogels.

For oral administration, the compounds may be administered in capsule, tablet, or bolus form. The capsules, tablets, and boluses are comprised of the active ingredient in combination with an appropriate carrier vehicle such as starch, talc, magnesium stearate, or di-calcium phosphate. These unit dosage forms are prepared by intimately mixing the active ingredient with suitable finely-powdered inert ingredients including diluents, fillers, disintegrating agents, and/or binders such that a uniform mixture is obtained. An inert ingredient is one that will not react with the compounds or modulators and which is non-toxic to the animal being treated. Suitable inert ingredients include starch, lactose, talc, magnesium stearate, vegetable gums and oils, and the like. These formulations may contain a widely variable amount of the active and inactive ingredients depending on numerous factors such as the size and type of the animal species to be treated. The active ingredients are intimately mixed with these inert carriers by grinding, stirring, milling, or tumbling such that is the final composition contains from 0.001 to 5% by weight of the active ingredient.

The compounds may alternatively be administered parenterally via injection of a formulation consisting of the active ingredient dissolved in an inert liquid carrier. Injection may be either intramuscular, intraruminal, intratracheal, or subcutaneous. The injectable formulation consists of the active ingredient mixed with an appropriate inert liquid carrier. Acceptable liquid carriers include the vegetable oils such as peanut oil, cotton seed oil, sesame oil and the like as well as organic solvents such as solketal, glycerol formal and the like. As an alternative, aqueous parenteral formulations may also be used. The vegetable oils are the preferred liquid carriers. The formulations are suitably prepared by dissolving or suspending the active ingredient in the liquid carrier such that the final formulation contains from 0.005 to 10% by weight of the active ingredient.

Topical application of the compounds or modulators is possible through the use of a liquid drench or a shampoo containing the instant compounds or modulators as an aqueous solution or suspension. These formulations generally contain a suspending agent such as bentonite and normally will also contain an antifoaming agent. Formulations containing from 0.005 to 10% by weight of the active ingredient are acceptable. Preferred formulations are those containing from 0.0.1 to 5% by weight of the instant compounds.

All publications and patent applications mentioned in this specification are herein incorporated by reference to the same extent as if each individual publication or patent application was specifically and individually indicated to be incorporated by reference.

The invention now being fully described, it will be apparent to one of ordinary skill in the art that many changes and modifications can be made thereto without departing from the spirit or scope of the appended claims.

The invention also provides methods for screening compounds that may be useful in treating METH addiction as well as other drug addiction conditions. These methods may best be illustrated with the following series of Examples, which illustrate the screening of mirtazapine, and SDZ SER 082 found by the inventors to be useful in the practice of the present invention, and ketanserin, found to be ineffective for the proposed therapeutic treatment of METH disorders.

### EXAMPLES

### Example I

Methamphetamine-induced behavioral sensitization.

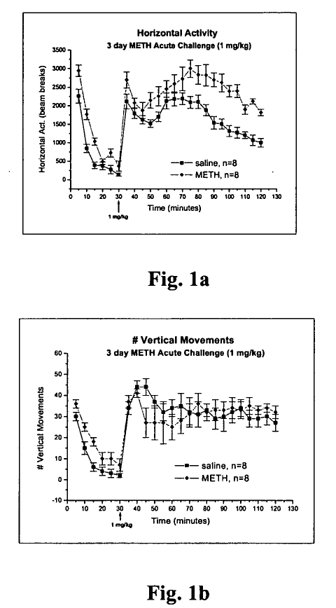

Automation of observational evaluations was accomplished using computerized small animal monitors (AccuScan Instr. Inc., Columbus, Ohio). The sensitization profiles quantified by the AccuScan monitors in rats following 5, once-daily days of METH treatment (2.5 mg/kg) were found to mirror that observationally described for similarly treated rats. One feature of drug-induced behavioral sensitization is heterogeneity of motor responses and the AccuScan monitors quantify numerous behavioral indices. This is a critical point for the motor profile is exquisitely related to METH dose, the drug history of the rat and observation time. After 3- or 31-days of withdrawal from repeated METH injections, rats were allowed to achieve baseline motor activity with a 30 min habituation period, challenged with 1 mg/kg sc METH and monitored for 90 min. As shown in FIGS. 1a-b, a “sensitized” motor response was clearly expressed in the METH-pretreatment group to the METH challenge at the 3-day withdrawal period for both Horizontal Activity and Stereotypy Counts (data not shown) and with slight early effects on Vertical Movements.

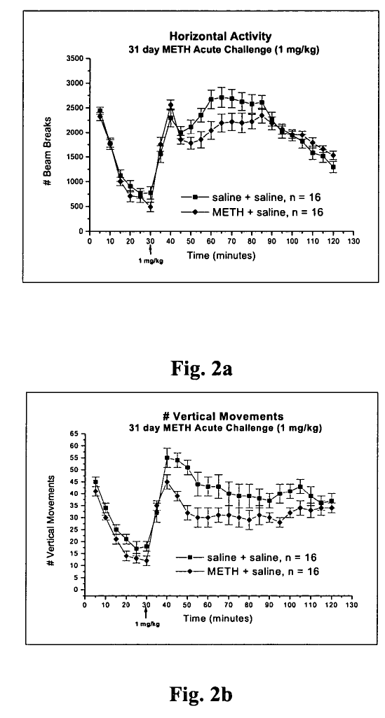

A METH challenge at 31 days post-repeated METH induced lower horizontal activity scores than those obtained after repeated saline (see FIGS. 2a-b). Vertical movements (i.e., up and down/rearing frequency) also were reduced throughout the recording period but the time spent in a rear or wall climb, as well as body movements while in a rear or wall climb were not diminished (data not shown). This distinct profile occurs when the rats show a preference to stand up on their hind limbs and remain vertical in a confined space. Thus, unique patterns of behavioral responses to an acute METH challenge emerge with longer withdrawal periods and it is likely that the neurobiological substrates that underlie the tardive emergence and maintenance of these behaviors may differ from those that underlie behaviors expressed following short term withdrawals.

Reversal of sensitization by 5-HT_{2A/2C} antagonists.

Multiple 5-HT_{2A/2C} antagonists with differing pharmacological profiles were tested for their ability to ameliorate METH-induced behavioral sensitization when administered after sensitization has developed. The antagonists, mianserin (1.0 and 2.5 mg/kg), mirtazapine (5 mg/kg) and ketanserin (1.0 mg/kg) were tested. The doses were selected based on the antagonists' ability to block 5-HT_{2A}- and 5-HT_{2C}-mediated activity and their pharmacokinetic profiles.

Using the METH pretreatment protocol described above in this Example I, the antagonists were given for 3 weeks (once daily, M-F) starting on withdrawal (w/d) day 3 in saline- or METH-pretreated rats and a METH acute challenge was tested on w/d day 30/31 (thus, the antagonist was largely cleared from the rat).

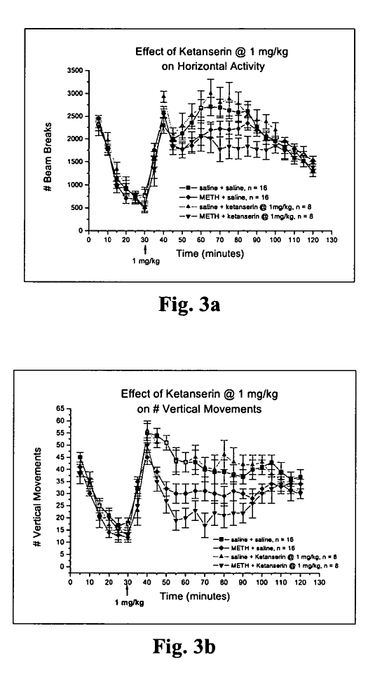

Ketanserin at 1 mg/kg is relatively selective for the 5-HT_{2A} receptor, while at higher doses (e.g., 5 mg/kg) ketanserin can also antagonize 5-HT_{2C} sites.[27-30] The inventors posed that if low dose ketanserin reverses METH-induced sensitization, then it can be suggested that selective 5-HT_{2A} blockade alone would be sufficient. Ketanserin, which is a 5-HT_{2A/2C} antagonist without antidepressant efficacy, also provided a useful comparison to mianserin and mirtazapine, 5-HT_{2A/2C} antagonists that are antidepressants. Representative results of the behavioral studies with ketanserin (1 mg/kg) are shown in FIGS. 3a-b. Ketanserin-treatment did not produce any locomotor effects on the saline-pretreated animals (i.e., the scores to the acute METH challenge were similar to the saline+saline pretreated rats) indicating no residual effect of the 3-week ketanserin treatment on locomotor endpoints. As shown, the response to a 1 mg/kg METH challenge in the METH-pretreated group was distinct from the saline-pretreated group, but unpredictably, ketanserin appeared to potentiate the effect on both horizontal activity and vertical movements suggesting an enhancement of the METH response by selective 5-HT_{2A} receptor blockade. These important findings paralleled the electrophysiological assessments from similarly treated rats (overviewed below).

Approximately 50% of METH addicts have a psychiatric diagnosis related to mood disorders and depression; a proportion that is twice as high as cocaine addicts.[31] It could be argued that antidepressants that have a high affinity for the 5-HT₂ receptor family, such as mianserin and mirtazapine, target METH-induced changes that are similar to those seen in depression. [32]

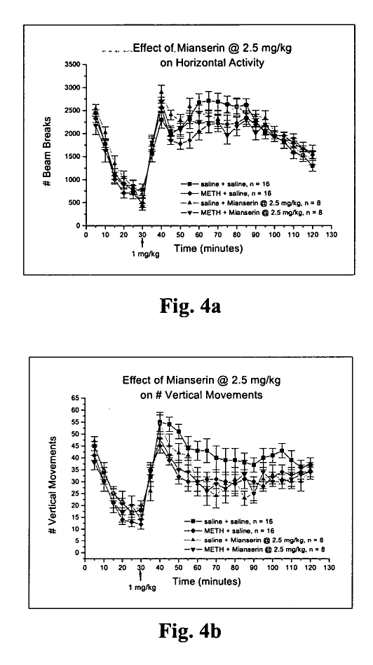

Mianserin is a 5-HT antagonist with high affinity for both the 5-HT_{2A} and 5-HT_{2C} receptor subtypes whose clinical safety has already been demonstrated. Evaluations of the ability of mianserin to influence the motor effects of METH were conducted. Data collected from mianserin-treated animals (daily 2.5 mg/kg M-F for 3 weeks) are shown in FIGS. 4a-b. Some attenuation of the METH response on horizontal activity was seen within the first 30 min of the 90 min behavioral assessment. However, the 3-week treatment of mianserin also appeared to have an independent effect on number of vertical movements regardless of METH or saline pretreatment group. No differences were seen between the saline+mianserin and the METH+mianserin pretreatment groups, and unexpectedly, the METH+saline animals generally were not distinguished from the METH+mianserin group. These findings together clearly establish a different pattern in behavioral responses between ketanserin (1 mg/kg) and mianserin (2.5 mg/kg) treated rats suggesting that differences in pharmacological profiles are critical to the adaptive behavioral changes evoked by repeated METH-treatments. It is noteworthy that total distance traveled, ambulatory time, vertical activity (early) and vertical time (early) indices of METH-induced sensitized behaviors also were attenuated by the 2.5 mg/kg dose of repeated mianserin pretreatments (data not shown).

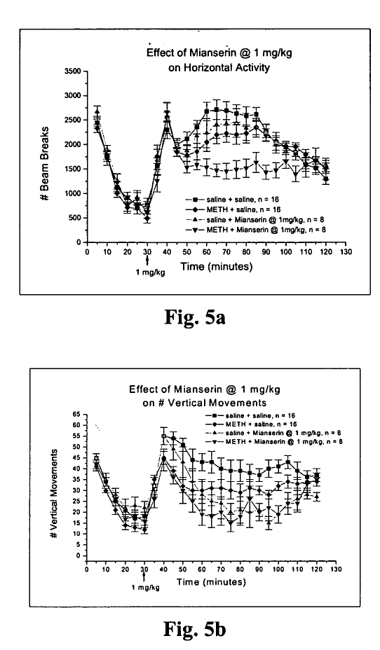

Analysis of the 1 mg/kg dose of mianserin provided a profile of motor endpoints more consistent with ketanserin suggesting a preferential influence of 5-HT_{2A} receptor blockade at this dose. (Note: mianserin's in vitro receptor selectivity profile suggests the differential between 5-HT_{2A}>5-HT_{2C}>5-HT₃ (2>5>8 nM, respectively), is too narrow, such that definitive conclusions are not possible.) These findings are summarized in FIGS. 5a-b. The low dose of mianserin further reduced the horizontal and vertical activity endpoints obtained in METH-pretreated rats, indicating a potentiation of the sensitized METH response, and thus indicating that mianserin was not suitable for use in the present invention. Interestingly as seen with the high dose the effect on 3-week mianserin treatment on vertical movements in both the METH and saline treatment groups was very prominent suggesting an underlying adaptation in behavioral responding to the mianserin treatment by itself, also indicating that mianserin may not be suitable for use in the present invention.

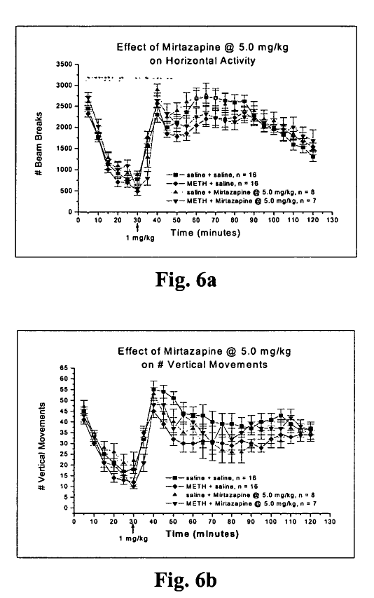

Mirtazapine (daily 5 mg/kg×15 days, given M-F) provided the greatest overall attenuation of METH-sensitized responses (FIGS. 6a-b). As seen with the other tested antagonists, the mirtazapine+saline group did not differ from the saline+saline pretreated rats, but in contrast to both ketanserin and the low dose of mianserin, mirtazapine did not potentiate METH-induced sensitization for any of the assessed behaviors.

A slight attenuation of horizontal activity was seen within the first 60 min after the acute METH challenge, followed by an attenuation of the vertical movement suppression seen in METH-pretreated rats. Moreover, mirtazapine attenuated the decreases in total distance traveled and time spent ambulating that were induced by METH pretreatments (data not shown), indicative of an attenuation of the preference to remain vertical in a confined space also seen in METH-sensitized rats. These results indicated that the distinct pharmacological profile of mirtazapine may be desirable in attenuating the overall METH-sensitized behavioral changes that appear to manifest from underlying long-lasting biochemical and electrophysiological changes of particular neuronal systems. Review of the published literature regarding underlying pharmacological profiles suggests that mirtazapine has the following rank ordered affinity for several CNS receptors: H1>5-HT_{2A}≧-5-HT_{2C}≧5-HT₃. This may suggest an increase in H1 activity as well as 5-HT_{2C} and 5-HT₃ in relationship to 5-HT_{2A} affinity may be beneficial.

### Example II

Methamphetamine-induced associative learning, as assessed by place conditioning.

Studies described in the studies of Example I helped identify mirtazapine as a 5-HT_{2A/2C} antagonist with a profile most likely to meet our objective of ameliorating the neuronal and behavioral effects of repeated METH exposure. To enhance the validity of the behavioral model of addiction, and thus to promote the ability of the rodent work to translate into the human condition, we developed a novel approach to assessing the behavioral consequences that incorporates a means to quantify the incentive properties of METH. As enhanced attribution of salience to METH is a key feature that drives a drug-withdrawn addict to again seek drug and to relapse into drug taking, the therapies of the present invention address this very feature. Incentive salience can be measured in rats using place conditioning procedures. Akin to the craving for METH that is evoked in human addicts when they are exposed to people, places or things that they previously associated with their drug-taking, place conditioning tasks quantifies the rat's desire to associate with an environmental cue that had been previously paired with METH administration. The novel approach of the present invention allows the simultaneous assessment of conditioned place preference (CPP) and motor sensitization (as in Example I), and thus, offers a unique and powerful means to discriminate drug efficacies for mitigating these two important (but divergent) models of addiction.

The CPP box (Accuscan, Columbus, Ohio) consists of three Plexiglas compartments, each with distinct visual and tactile cues (on one side, patterned floor with object attached and horizontally striped wall; on the opposite side, smooth floor with no object and vertically striped walls; center—uniformly white floor and walls). The center compartment can be separated from the left and right compartments by removable guillotine doors. Motor activity in three dimensional space, and time spent, in each compartment is detected by two sets of photobeams set at different heights from the floor. Drug conditioning is performed by administering METH to the rat while it is in one compartment, and on the alternating day, saline is administered while the rat is confined to the opposite compartment. Confinement was achieved by blocking access to other compartments using the guillotine door. The center compartment is not seen by the rat during conditioning, and all conditioning sessions last for 45 min.

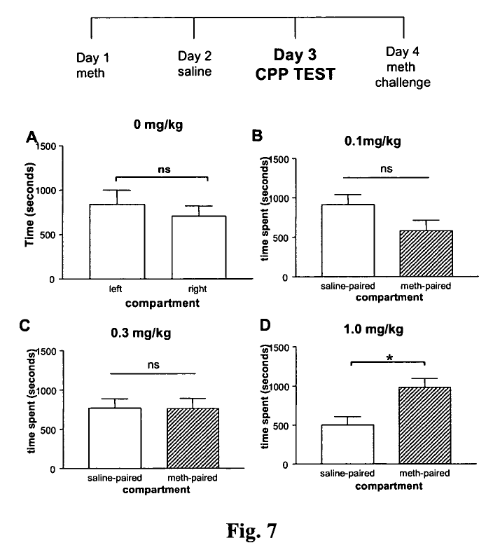

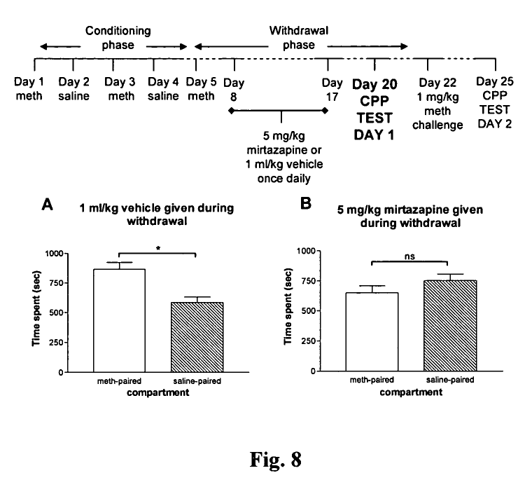

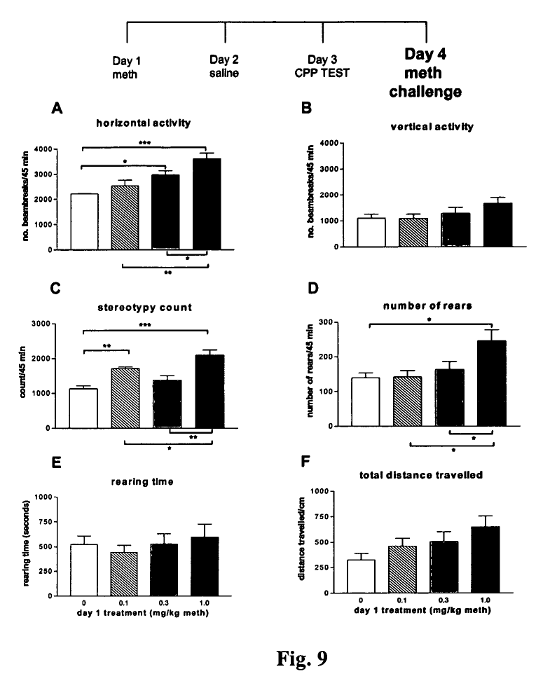

The inventors have determined that the number of METH-pairings, and the dose of METH, dictate the strength of the drug-environment association (i.e., the magnitude of salience attribution). The test for CPP is determined in a METH-free state (i.e., at least 48 hr after that last METH pairing), at which time the rats are placed in the center compartment and allowed free access to all compartments for 30 min, and the time spent in each compartment is the index of preference. The inventors have determined that the number of METH-pairings, and the dose of METH, dictate the strength of the drug-environment association (i.e., the magnitude of salience attribution) and thus the amount of time spent in the compartment previously paired with METH (FIG. 7), as well as persistence of this effect (i.e., how long it lasts; FIG. 8A). METH-induced motor sensitization can be assessed in several ways, including assessing the capacity of the rat to express an enhanced motor response to an acute METH challenge (FIGS. 9 & 10A). As these studies demonstrated for the first time that the METH dose which induces motor sensitization and CPP differ (e.g., compare FIG. 8A with FIG. 9C), the simultaneous monitoring of the two behavioral endpoints for the effects of 5-HT_{2A/2C} antagonists will allow identification of novel treatments.

Reversal of place preference by 5-HT_{2A/2C} antagonists.

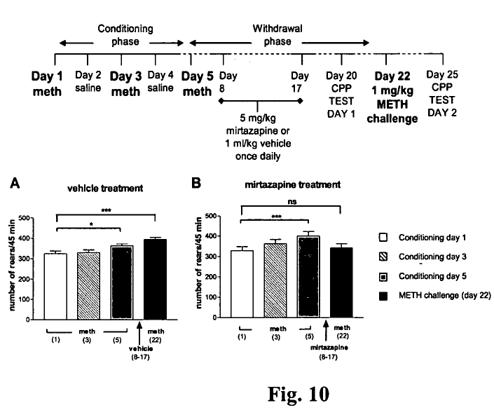

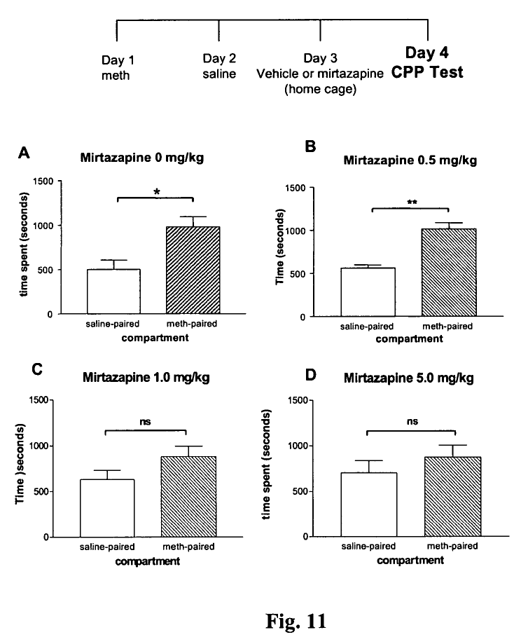

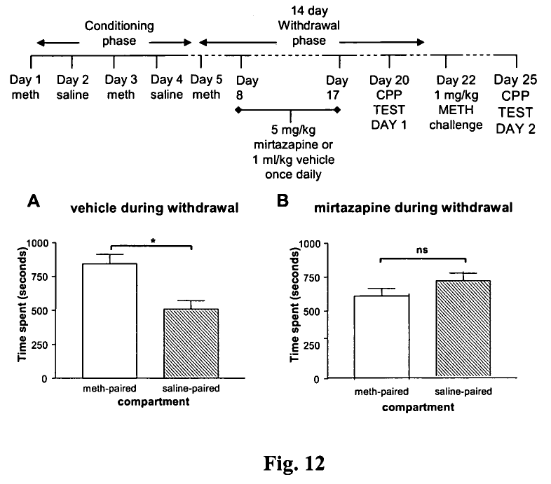

The inventors have demonstrated for the first time, that mirtazapine can completely reverse the associative learning process that compels rats to demonstrate preference to the place previously paired with METH (FIG. 8B) and associated motor sensitization (FIG. 10B). To efficiently apply this approach to testing of 5-HT_{2A/2C} antagonists, we determined that single pairing protocols of the CPP paradigm can be used to predict efficacy outcomes of 5-HT_{2A/2C} antagonists in long-term tests on these drugs evaluating their capacity to nullify the persistent enhanced salience induced by METH. This is illustrated by comparing FIG. 8B with FIG. 11, where a dose-response analysis was conducted to determine the single injection dose(s) that would reverse CPP expression to a single pairing of METH (FIG. 11) and repeated injections of the effective dose also reversed the rats' demonstration of preference that normally would persist for weeks after multiple METH pairings (FIG. 8B). Remarkably, this later treatment also renders ineffective the ability of subsequent METH pairings to induce CPP (FIG. 12). Thus, these protocols should help predict the capacity of novel therapeutic targets to halt a common problem of drug relapse, i.e., the ability of re-exposure to drugs like METH to immediately reinstate their abuse.

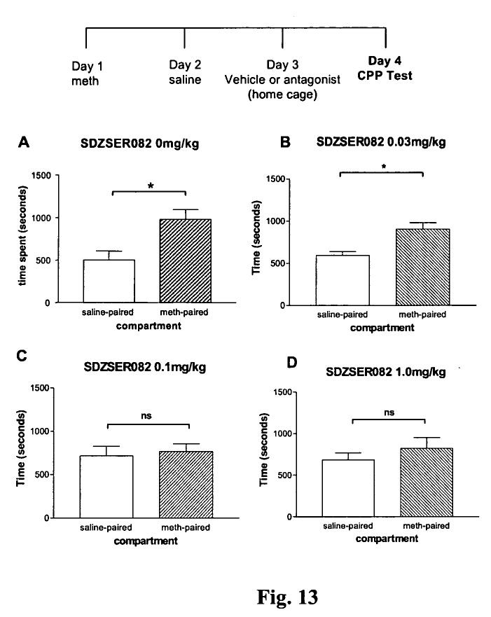

Mirtazapine has a high affinity for both the 5-HT_{2A} and the 5-HT_{2C} receptor subtypes (see Definitions section). We have demonstrated for the first time that these two subtypes differentially regulate METH-induced CPP, and thus, likely will play different therapeutic roles in METH addiction. SDZSER082, is a highly selective 5-HT_{2C} receptor antagonist, and it reverses METH-induced CPP in a dose-dependent fashion (FIG. 13). These data demonstrate the utility of the single METH pairing CPP protocol to distinguish pharmacologics with very subtle profile differences.

### Example III

Gene transcription as brain region-specific markers for the effects for METH withdrawal and their reversal with 5-HT_{2A/2C} antagonists.

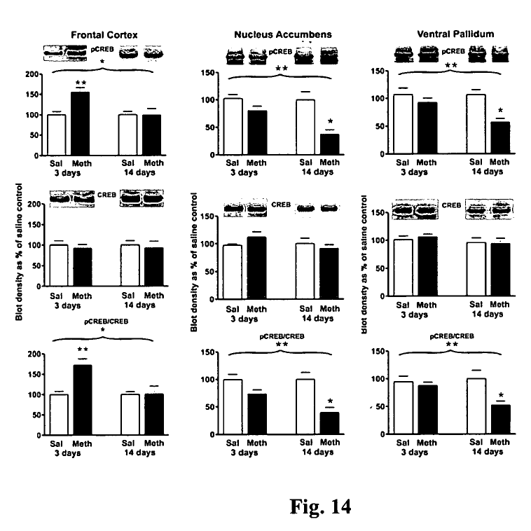

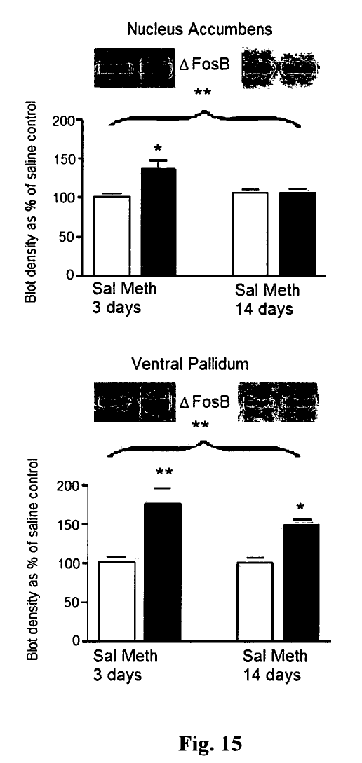

Receptor-mediated changes in cellular Ca²⁺ and cAMP can give rise to persistent neuroplastic changes through modulation of transcription factors and ensuing changes in gene transcription. Amphetamine and cocaine modify gene transcription through the phosphorylation and activation of CREB[33], or through ΔFosB, the level of which has been shown to be increased after chronic cocaine. To investigate whether METH also modifies the activity of CREB and levels of ΔFosB, we assayed for pCREB, CREB and ΔFosB (with Western blot techniques) in the frontal cortex, nucleus accumbens and ventral pallidum, harvested 3 and 14 days after repeated METH (2.5 mg/kg). The nucleus accumbens and ventral pallidum showed a decrease in the activation state of CREB (pCREB/CREB ratio) (FIG. 14) at 14 days withdrawal. In contrast, the frontal cortex showed elevated levels of pCREB at 3 days withdrawal (FIG. 14). Levels of ΔFosB (FIG. 15) were unchanged in the cortex, but elevated in both the accumbens and pallidum at 3 days withdrawal and this increase persisted to 14 days in the ventral pallidum. These data support the hypothesis that METH-induced sensitization is associated with brain region, and time-dependent changes in pCREB and ΔFosB, giving rise to a dynamic pattern of genetic transcriptional control. Moreover, these data are consistent with a hypothesis of Ca²⁺-related signaling, such as that mediated via activation of 5-HT_{2A/2C}-receptors, contributing to these events.

The following were assessed using Western blotting technology: a) phosphorylated CREB (pCREB)—to ascertain the level of phosphorylated (activated) CREB; b) total CREB—to determine whether changes in pCREB are related to changes in total cellular CREB protein, and c) ΔFosB—to ascertain the level of ΔFosB antigen (37 kDa). ΔFosB analysis was established and initial results with the mirtazapine treatment group confirmed loss of increased ΔFosB early signal in the nucleus accumbens by day 31 as consistent with the above examples (data not shown).

### Example IV

Electrophysiology of methamphetamine-induced cellular sensitization and the involvement of 5-HT_{2A/2C} receptors.

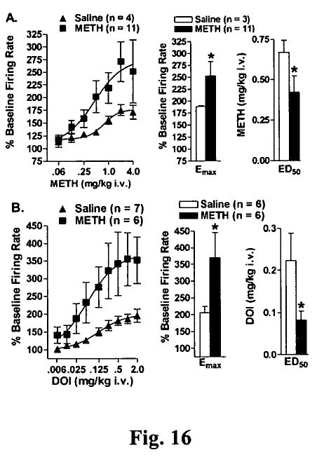

Based on the biochemical results and because the ventral pallidum contains one of the highest concentrations of 5-HT in the brain, electrophysiological evaluations of this region in rats behaviorally sensitized to METH were conducted (with a 5-day once daily treatment of 2.5 mg/kg). The acute challenge was intravenously administered METH (via a tail vein cannula) as previously used by T. C. Napier's lab ^{e.g., [}34] where the drug is given in a cumulative dosing fashion such that each dose, administered in 2 min-intervals, essentially doubles the previous dose. METH was tested using a range of 0.06-4.0 mg/kg in saline vehicle. This popular dosing paradigm allows for comparisons of potency and efficacy and reveals cellular changes in chloral hydrate-anesthetized rats that were previously sensitized to other psychomotor stimulants.[35-38] Moreover, this approach establishes if systemic administration of METH, in doses that include those producing behavioral sensitization, are sufficient promote sensitized cellular responding and thus allows for direct comparisons of cellular responding to behavioral outcomes. After a 3-day withdrawal, the ability of intravenously administered METH or 1-(2,5-dimethoxy-4-iodophenyl)-2-aminopropane (DOI) to increase ventral pallidal cell firing was enhanced in chloral hydrate-anesthetized rats, as shown by a leftward shift in the dose-response curve with a decrease in potency and an increase in response efficacy for both agonists (FIG. 16) and this occurred without any changes in the portion of neurons showing a rate increase or decrease to the acute METH challenge.

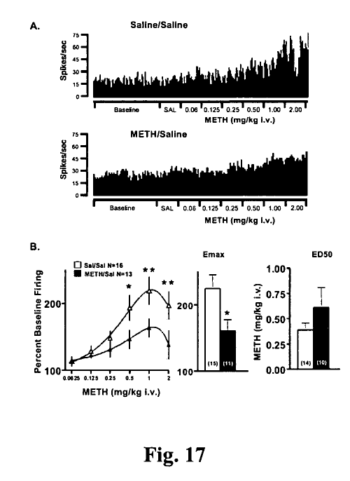

Electrophysiological assessment of the ventral pallidum was also conducted 30 days after repeated METH. Two hundred and twenty electrophysiological experiments were conducted, from treatment groups comprised of rats receiving METH or Saline (Sal) once daily for 5 days followed by mirtazapine (Mirt), ketanserin (Ket), or its Sal vehicle (veh) for 15 days, and tested 31 days after the last METH (or vehicle) injection. For each experiment, an i.v. METH dose-response curve (METH was tested using a range of 0.06-2.0 mg/kg in saline vehicle) was generated for one ventral pallidal neuron per rat at 30 days withdrawal from repeated METH. Based on the analysis of curves where there was an excitatory effect of i.v. METH on ventral pallidal neurons (171 animals), there was a diminished excitatory effect of i.v. METH in the ventral pallidum of rats that were pretreated with METH only. This is illustrated by both the curves and corresponding Emax data in FIG. 17. This decrease only at long-term withdrawal mirrors the reduction seen in pallidal pCREB only after 14, but not at 3 day withdrawal from repeated METH (see Example III). Table 1 illustrates the Emax data obtained from the excitatory dose response curves generated from the rats treated with ketanserin or mirtazapine after METH withdrawal. These data indicate a reversal by mirtazapine of the effect of repeated METH on the response of ventral pallidal neurons to an acute METH challenge after 30 days withdrawal.

TABLE 1
Emax data obtained from ventral pallidal dose response
curves to i.v. METH in chloral hydrate anaesthetized
rats. ANOVA followed by Dunnett's post hoc.

| Repeated | Emax of Firing Rate |
|---|---|
| PreTreatment Group | Increases of Acute Meth |
| Days 1-5 + Days 15-30 | (% Baseline) |
| Sal + Veh | 225% ± 20% (n = 15) |
| Sal + Mirt | 240% ± 44% (n = 5) |
| METH + Veh | 161% ± 16% (n = 11) * |
| METH + Mirt | 251% ± 21% (n = 5) |
| METH + Ket | 177% ± 18% (n = 4) |

* P < 0.05 as compared to saline + vehicle.

### Example V

Methods: Immunoblot Assays

In METH-sensitized rats, transcription factor regulation (activation of CREB and ΔFos B levels) can be determined in accordance with the present invention for a survey of brain regions known to be involved in addictive behaviors. The ability of the compounds to reverse these effects will be ascertained. Those regions in which the test compound is able to reverse the biochemical profiles mediated by repeated METH treatments will then be evaluated electrophysiologically. The experiments will ascertain, at the level of cell function, whether the antagonist effectively restores the METH altered brain to normal.

Using immunoblotting techniques, CREB, pCREB and ΔFos B changes are monitored that accompany the long-term behavioral sensitization caused by METH and the effect of drug treatment on these markers. Brain regions can be analyzed are frontal cortex, dorsal striatum, nucleus accumbens, ventral pallidum, globus pallidus, and amygdala. (Table 2).

TABLE 2
Biochemical assessment of the effectiveness novel agents.

| Chronic | w/d Days 1-20 | w/d Day 31 | |
|---|---|---|---|
| Treatment | Antagonist | Acute Challenge | No. Rats |
| METH | Saline | METH | 8 |
| Saline | Saline | METH | 8 |
| METH | Test Compound | METH | 8 |
| Saline | Test Compound | METH | 8 |

Animals are killed by decapitation without anesthesia. Brain regions (frontal cortex, nucleus accumbens, dorsal striatum, ventral pallidum, globus pallidus and amygdala) are rapidly dissected over ice and snap frozen on dry ice. Tissue is stored at −80° C. until prepared and assayed as described below.

Membrane preparation. Tissue is homogenized in 20 volumes of 25 mM HEPES-TRIS, pH 7.4, containing 1 mM EGTA, 1 mM EDTA, 100 nM okadaic acid, 1 mM sodium orthovanadate and 100 uM PMSF and further processed for SDS-PAGE and western blotting.

SDS-PAGE and immunoblotting. In accordance with the present invention, samples of membrane protein are prepared and run on 10% BIS-TRIS resolving gels in MOPS running buffer (NuPage Electrophoresis System; Invitrogen; Carlsbad, Calif.). 20 μg samples of protein are loaded per lane. Proteins are then electrophoretically transferred onto a PVDF membrane (transfer buffer: 25 mM Tris, 192 mM glycine, 20% methanol, pH 8.0). Non-specific protein binding sites on the membrane are blocked by incubation at room temperature for 1 hr in blocking buffer (Tris-buffered saline containing 0.05% Tween-20 and 5% instant non-fat dry milk). After washing twice for 5 min each in Tris-buffered saline (TBS; 25 mM Tris-HCl, pH 7.4, 140 mM NaCl, 0.02% sodium azide, 0.05% Tween 20), the membrane is incubated in fresh blocking solution containing the desired primary antibody as directed by the supplier. Primary antibodies to be used are rabbit anti-phospho(Ser133)CREB (1:3000; Cell Signaling; Beverly, Mass.), rabbit anti-CREB (1:3000; Cell Signaling Technology; Beverly, Mass.), rabbit anti-FosB (1:2000; Santa Cruz Biotechnology; Santa Cruz, Calif.). After 3 washes (20 min each) with TBST, the membrane is incubated with alkaline-phosphatase conjugated secondary antibody (1:20,000 dilution; Promega) in blocking buffer for 1 hr at room temperature. Immunoreactive bands are visualized using the enhanced chemiluminescence method (ImmunStar; BioRad). Optical density of immunoreactive bands will be analyzed. Brain regions will be analyzed for each independent parameter measured (e.g., pCREB, total CREB, ΔFosB 37 kDa). Two-way ANOVA (treatment×time) will be used to compare between METH- and saline-treated rats.

### Example VI

Methods: Electrophysiology methods.

In one aspect of the present invention, electrophysiological assessment of METH-induced sensitization in the brain regions provide a functional correlate, at the cellular level, to the previously described biochemical evaluations. These studies are anticipated to determine if systemic administration of METH and 5-HT ligands, in doses that are similar to those producing behavioral sensitization, are sufficient promote sensitized cellular responding. The test compounds are applied only to the local environment around the recorded neurons, and allow for correlations to the cellular effects ascertained in the biochemical experiments.

Overview of experimental design. Rats will receive daily injections of METH or saline for 5 days and their motor behavior will be quantified on days 1 and 5. Thirty-one days after the last METH injection, the rats will be anesthetized with chloral hydrate and single cell spiking will be isolated from the brain region of interest. Representing an input and output pathway of the limbic system, respectively, both the nucleus accumbens [39;40] and the ventral pallidum[41;42] respond to 5-HT agonists, and both show cellular sensitization to psychomotor stimulants. Using two regions as examples, the following tables and accompanying tests overview a proposed scenario for treatment groups.

TABLE 3
Acute challenge (AC) in chloral hydrate-anesthetized rats.

| Proposed | Chronic | 31 day w/d i.v. or |
|---|---|---|
| Brain Region | Treatment | iontophoretic AC |
| Ventral pallidum | METH | METH; test drugs |
| Ventral pallidum | Saline | METH; test drugs |
| N. accumbens | METH | METH; test drugs |
| N. accumbens | Saline | METH; test drugs |

Protocol 1. For the i.v. acute challenge (A/C) (Table 3), a complete dose-response curve can be generated for each neuron tested, and only one neuron will be tested per rat. The AC ligand will be administered via a tail vein cannula in a cumulative dosing fashion such that each dose, given in 2 min-intervals, essentially doubles the previous dose. (For example, METH will be tested using a range of 0.06-4.0 mg/kg in saline vehicle.) This popular dosing paradigm allows for comparisons of potency and efficacy, showing changes in neuronal sensitivity to various agonists following repeated amphetamine or cocaine treatments, ^{e.g.,} [35-38] and as used by T. C. Napier's lab^{e.g.,}[34;41;43-46]

Protocol 2. To determine if the local receptor environment is altered, agonists will be discretely applied onto the recorded neuron using microiontophoresis. An iontophoretic current (“dose”)/response curve will be generated for each agonist. As previously shown by T. C. Napier and others[34;34-36;47-58] the magnitude of the iontophoretic ejection current correlates to the magnitude of the evoked response, and this approach provides a rapid efficient method to compare the cellular receptor-mediated effects of test compounds on each recorded neuron (Table 4).

TABLE 4
The effect of novel agents on METH-induced responding in rats

| | Chronic | w/d Days 1-10 | w/d Day31, i.v. or |
|---|---|---|---|
| Brain Region | Treatment | Antagonist | iontophoretic AC |
| Ventral pallidum | METH | Saline | METH; test drugs |
| Ventral pallidum | Saline | Saline | METH; test drugs |
| Ventral pallidum | METH | Test compound | METH; test drugs |
| Ventral pallidum | Saline | Test compound | METH; test drugs |
| N. accumbens | METH | Saline | METH; test drugs |
| N. accumbens | Saline | Saline | METH; test drugs |
| N. accumbens | METH | Test compound | METH; test drugs |
| N. accumbens | Saline | Test compound | METH; test drugs |

Electrophysiological recording procedures. Single barrel glass pipettes, purchased (A-M Systems, Inc.) preloaded with a glass fiber will be heat-pulled and the tips broken back to 2 μm. The recording pipette will be filled with a 0.5 M sodium acetate, 2% Pontamine sky blue solution. Extracellularly-recorded action potentials will be amplified and displayed on a Tektronix storage oscilloscope. Individual spikes will be isolated with a Fintronics amplitude analyzer/audio analyzer with the window output fed into an IBM compatible computer. In house electrophysiological software will be used for on-line data acquisition, generation of real time and interspike interval histograms, and subsequent analysis of intraveneously administered drugs. After encountering a neuron, firing will be monitored for at least 5 min and the action potential characteristics and firing pattern will be ascertained. For the microiontophoretic experiments, a method routinely used by T. C. Napier, ^{e.g.,} [34;34;51-58] will be employed. Here, glass multibarrel pipettes (A-M Systems, Inc.) will be will be heat-pulled, tips broken back to 12 μm, and glued in parallel with a recording microelectrode. The center barrel will be filled with 2 M NaCl (15-25 MΩ) to be used for automatic balancing of the current at the tip of the pipette. The side barrels will be filled with various combinations of test ligands (in 10 mM base, pH adjusted to 4-4.5; 20-60 MΩ; using 5-120 nA, this expels the ligands in nM concentrations into the local milieu of the neuron) or their vehicle solutions. A six channel current generator and programmer (Fintronics) will be used for microiontophoretic ejection (using +5 to +80 nA) and retention (using −10 nA) of drugs from the pipettes. Appropriate current and vehicle controls (which previously have been shown to not induce changes in spiking ^{e.g.,} [57] will be performed.

Histology. At the end of the electrophysiological experiments, pontamine sky blue will be deposited at the electrode tip with an anionic current. The brains are removed, stored in 10% formalin-30% sucrose and then cut on a freezing microtome (40 μm coronal sections). Sections will be mounted on gel-coated slides and stained with cresyl violet. Recording sites will be reconstructed onto a standard map of the rat brain.

Statistical evaluations. Linear regression analysis of the i.v. dose, or the ejection current magnitude, versus firing rate will be used to ascertain if the magnitude of the i.v. dose or the microiontophoretic ejection current is related to the response magnitude of the recorded neurons. This treatment-effect relationship is considered to have occurred if the slope of the line was significantly different from zero. Third order polynomials will be fit to each neuron's response to multiple treatment applications. Those where r²≧0.7 are used to determine the maximal effect (Eₘₐₓ) of an agonist and the current or dose necessary to produce 50% of the maximal effect (ED₅₀, respectively). Agonist Eₘₐₓ and Ecur₅₀ or ED₅₀ in the various treatment conditions will be compared using ANOVA, with Newman-Keuls pairwise post hoc evaluations; using P<0.05.

Citations
1. Ito, K., Ohmori, T., Abekawa, T., and Koyama, T., Clonazepam prevents the development of sensitization to methamphetamine, Pharmacol. Biochem. Behav., 58 (1997) 875-879.
2. Szumlinski, K. K., Balogun, M. Y., Maisonneuve, I. M., and Glick, S. D., Interactions between iboga agents and methamphetamine sensitization: studies of locomotion and stereotypy in rats, Psychopharmacology (Berl), 151 (2000) 234-241.
3. Itzhak, Y. and Martin, J. L., Effect of riluzole and gabapentin on cocaine- and methamphetamine-induced behavioral sensitization in mice, Psychopharmacology (Berl), 151 (2000) 226-233.
4. Ohmori, T., Abekawa, T., and Koyama, T., Scopolamine prevents the development of sensitization to methamphetamine, Life Sci., 56 (1995) 1223-1229.
5. Nishikawa, T., Mataga, N., Takashima, M., and Toru, M., Behavioral sensitization and relative hyperresponsiveness of striatal and limbic dopaminergic neurons after repeated methamphetamine treatment, Eur. J. Pharmacol., 88 (1983) 195-203.
6. Kodama, M., Akiyama, K., Ujike, H., Shimizu, Y., Tanaka, Y., and Kuroda, S., A robust increase in expression of arc gene, an effector immediate early gene, in the rat brain after acute and chronic methamphetamine administration, Brain Res, 796 (1998) 273-283.
7. Tzschentke, T. M., Measuring reward with the conditioned place preference paradigm: a comprehensive review of drug effects, recent progress and new issues, Prog. Neurobiol., 56 (1998) 613-672.
8. Walsh, S. L. and Cunningham, K. A., Serotonergic mechanisms involved in the discriminative stimulus, reinforcing and subjective effects of cocaine, Psychopharmacology (Berl), 130 (1997) 41-58.
9. Tran-Nguyen, L. T., Baker, D. A., Grote, K. A., Solano, J., and Neisewander, J. L., Serotonin depletion attenuates cocaine-seeking behavior in rats, Psychopharmacology (Berl), 146 (1999) 60-66.
10. Satel, S. L., Krystal, J. H., Delgado, P. L., Kosten, T. R., and Charney, D. S., Tryptophan depletion and attenuation of cue-induced craving for cocaine, Am. J. Psychiatry, 152 (1995) 778-783.
11. Davidson, C., Lee, T. H., Xiong, Z., and Ellinwood, E. H., Ondansetron given in the acute withdrawal from a repeated cocaine sensitization dosing regimen reverses the expression of sensitization and inhibits self-administration, Neuropsychopharmacology, 27 (2002) 542-553.
12. Davidson, C., 5-H2 receptor antagonists given in the acute withdrawal from daily cocaine injections can reverse established sensitization., Eur. J. Pharmacol, (2002) 255-263.
13. Galloway, G. P., Newmeyer, J., Knapp, T., Stalcup, S. A., and Smith, D., A Controlled Trial of Imipramine for the Treatment of Methamphetamine Dependece, Journal of Substance Abuse Treatment, 13 (1996) 493-497.
14. Galloway, G. P., Newmeyer, J., Knapp, T., Stalcup, S. A., and Smith, D., Imipramine for the treatment of cocaine and methamphetamine dependence, Journal of Addictive Diseases, 13 (1994) 201-216.
15. Fletcher, P. J., Korth, K. M., and Chambers, J. W., Of 5,7-dihydroxytryptamine does not alter d-amphetamine self-administration across different schedule and access conditions, Psychopharmacology (Berl), 146 (1999) 185-193.
16. Parsons, L. H. and Justice, J. B., Jr., Serotonin and dopamine sensitization in the nucleus accumbens, ventral tegmental area, and dorsal raphe nucleus following repeated cocaine administration, J. Neurochem., 61 (1993) 1611-1619.
17. Carroll, M. E., Lac, S. T., Asencio, M., and Kragh, R., Intravenous cocaine self-administration in rats is reduced by dietary L-tryptophan, Psychopharmacology (Berl), 100 (1990) 293-300.
18. Hoyer, D. Functional correlates of serotonin 5-HT1 recognition sites. J. Recept. Res. 8, 59-71. 1988.
19. Kelder, J., Funke, C., De Boer, T., Delbressine, L., Leysen, D., and Nickolson, V., A comparison of the physicochemical and biological properties of mirtazapine and mianserin, J. Pharm. Pharmacol, 49 (1997) 403-411.
20. Nozulak, J., Kalkman, H. O., Floersheim, P., Hoyer, D., Schoeffter, P., and Buerki, H. R., (+)-cis-4,5,7a,8,9,10,11,11a-octahydro-7H-10-methylindolo[1,7-bc][2,6]-naphthyridine: a 5-HT2C/2B receptor antagonist with low 5-HT2A receptor affinity, J. Med. Chem., 38 (1995) 28-33.
21. Filip, M., Role of serotonin (5-HT)₂ receptors in cocaine self-administration and seeking behavior in rats, Pharmacol. Rep., 57 (2005) 35-46.
22. Ferreira, H. S., Oliveira, E., Faustino, T. N., Silva, E. C., and Fregoneze, J. B., Effect of the activation of central 5-HT2C receptors by the 5-HT2C agonist mCPP on blood pressure and heart rate in rats, Brain Res., 1040 (2005) 64-72.
23. Dave, K. D., Harvey, J. A., and Aloyo, V. J., A novel behavioral model that discriminates between 5-HT2A and 5-HT2C receptor activation, Pharmacol. Biochem. Behav., 72 (2002) 371-378.
24. Ouagazzal, A., Grottick, A. J., Moreau, J., and Higgins, G. A., Effect of LSD on prepulse inhibition and spontaneous behavior in the rat. A pharmacological analysis and comparison between two rat strains, Neuropsychopharmacology, 25 (2001) 565-575.
25. Sipes, T. E. and Geyer, M. A., DOI disruption of prepulse inhibition of startle in the rat is mediated by 5-HT(2A) and not by 5-HT(2C) receptors, Behav. Pharmacol., 6 (1995) 839-842.
26. Mora, P. O., Netto, C. F., and Graeff, F. G., Role of 5-HT2A and 5-HT2C receptor subtypes in the two types of fear generated by the elevated T-maze, Pharmacol. Biochem. Behav., 58 (1997) 1051-1057.
27. Mazzola-Pomietto, P., Aulakh, C. S., Wozniak, K. M., and Murphy, D. L., Evidence that m-chlorophenylpiperazine-induced hyperthermia in rats is mediated by stimulation of 5-HT2C receptors, Psychopharmacology (Berl), 123 (1996) 333-339.
28. Mazzola-Pomietto, P., Aulakh, C. S., Wozniak, K. M., Hill, J. L., and Murphy, D. L., Evidence that 1-(2,5-dimethoxy-4-iodophenyl)-2-aminopropane (DOI)-induced hyperthermia in rats is mediated by stimulation of 5-HT2A receptors, Psychopharmacology (Berl), 117 (1995) 193-199.
29. Dursun, S. M. and Handley, S. L., Similarities in the pharmacology of spontaneous and DOI-induced head-shakes suggest 5HT2A receptors are active under physiological conditions, Psychopharmacology (Berl), 128 (1996) 198-205.
30. Willins, D. L. and Meltzer, H. Y., Direct injection of 5-HT2A receptor agonists into the medial prefrontal cortex produces a head-twitch response in rats, Journal of Pharmacology and Experimental Therapeutics, 282 (1997) 699-706.
31. Copeland, A. L. and Sorensen, J. L., Differences between methamphetamine users and cocaine users in treatment, Drug Alcohol Depend., 62 (2001) 91-95.
32. Kosten, T. R., Markou, A., and Koob, G. F., Depression and Stimulant Dependence: Neurobiology and Pharmacotherapy, Journal of Nervous and Mental Disease, 186 (1998) 737-745.
33. Shaywitz, A. J. and Greenberg, M. E., CREB: A Stimulus-Induced Transcription Factor Activated by a Diverse Array of Extracellular Signals, Annual Reviews of Biochemistry, 68 (1999) 821-861.
34. Napier, T. C., Chrobak, J. J., and Yew, J., Systemic and microiontophoretic administration of morphine differentially effect ventral pallidum/substantia innominata neuronal activity, Synapse, 12 (1992) 214-219.
35. Henry, D. J., Greene, M. A., and White, F. J., Electrophysiological effects of cocaine in the mesoaccumbens dopamine system: Repeated administration, J. Pharmacol. Exp. Ther., 251 (1989) 833-839.
36. Henry, D. J. and White, F. J., The persistence of behavioral sensitization to cocaine parallels enhanced inhibition of nucleus accumbens neurons, J. Neurosci., 15 (1995) 6287-6299.
37. Wolf, M. E., White, F. J., Nassar, R., Brooderson, R. J., and Khansa, M. R., Differential development of autoreceptor subsensitivity and enhanced dopamine release during amphetamine sensitization, J. Pharmacol. Exp. Ther., 264 (1993) 249-255.
38. Gao, W. Y., Lee, T. H., King, G. R., and Ellinwood, E. H., Alterations in baseline activity and quinpirole sensitivity in putative dopamine neurons in the substantia nigra and ventral tegmental area after withdrawal from cocaine pretreatment, Neuropsychopharmacology, 18 (1998) 222-232.
39. White, F. J., Henry, D. J., Hu, X.-T., Jeziorski, M., and Ackerman, J. M., Electrophysiological effects of cocaine in the mesoaccumbens dopamine system. In: Lakoski, J., Galloway, M. P., White, F. J. (Eds.), Cocaine: pharmacology, physiology and clinical strategies, CRC, Boca Raton, 1992, pp. 261-293.
40. White, S. R., Duffy, P., and Kalivas, P. W., Methylenedioxymethamphetamine depresses glutamate-evoked neuronal firing and increases extracellular levels of dopamine and serotonin in the nucleus accumbens in vivo, Neuroscience, 62 (1994) 41-50.
41. Heidenreich, B. A. and Napier, T. C., Effects of serotonergic 5-HT1A and 5-HT1B ligands on ventral pallidal neuronal activity, Neuroreport, 11 (2000) 2849-2853.
42. Bengtson, C. P. and Osborne, P. B., Electrophysiological properties of anatomically identified ventral pallidal neurons in rat brain slices, Ann N.Y. Acad Sci, 877 (1999) 691-694.
43. Heidenreich, B. A., Mailman, R. B., Nichols, D. E., and Napier, T. C., Partial and full dopamine D1 agonists produce comparable increases in ventral pallidal neuronal activity: Contribution of endogenous dopamine, J. Pharmacol. Exp. Ther., 273 (1995) 516-525.
44. Johnson, P. I. and Napier, T. C., Contribution of the nucleus accumbens to cocaine-induced responses of ventral pallidal neurons, Synapse, 22 (1996) 253-260.
45. Maslowski, R. J. and Napier, T. C., Dopamine D₁ and D2 receptor agonists induce opposite changes in the firing rate of ventral pallidal neurons., Eur. J. Pharmacol, 200 (1991) 103-112.
46. Maslowski, R. J. and Napier, T. C., Effects of D1 and D2 antagonists on apomorphine-induced responses of ventral pallidal neurons., Neuroreport, 2 (1991) 451-454.
47. Henry, D. J. and White, F. J., Repeated cocaine administration causes persistent enhancement of D1 dopamine receptor sensitivity within the rat nucleus accumbens, J Pharmacol Exp Ther., 258 (1991) 882-890.
48. Henry, D. J. and White, F. J., Electrophysiological correlates of psychomotor stimulant-induced sensitization, Ann N.Y. Acad Sci, 654 (1992) 88-100.
49. Henry, D. J. and White, F. J. Repeated cocaine administration causes persistent enhancement of D1 dopamine receptor sensitivity within the rat nucleus accumbens. Journal of Pharmacology and Experimental Therapeutics 258, 882-890. 1998.
50. White, F. J., Hu, X.-T., and Henry, D. J., Electrophysiological effects of cocaine in the rat nucleus accumbens: Microiontophoretic studies, J. Pharmacol. Exp. Ther., 266 (1993) 1075-1084.
51. Chrobak, J. J. and Napier, T. C., Opioid and GABA modulation of accumbens-evoked ventral pallidal activity, J. Neural Trans., 93 (1993) 123-143.
52. Johnson, P. I. and Napier, T. C., GABA- and glutamate-evoked responses in the rat ventral pallidum are modulated by dopamine, Eur. J. Neurosci, 9 (1997) 1397-1406.
53. Johnson, P. I. and Napier, T. C., Morphine modulation of GABA- and glutamate-induced changes of ventral pallidal neuronal activity, Neuroscience, 77 (1997) 187-197.
54. Mitrovic, I. and Napier, T. C., Electrophysiological demonstration of μ, δand kappa opioid receptors in the ventral pallidum, J. Pharmacol. Exp. Ther., 272 (1995) 1260-1270.
55. Mitrovic, I. and Napier, T. C., Interactions between the mu opioid agonist DAMGO and substance P in regulation of the ventral pallidum, Synapse, 23 (1996) 142-151.
56. Mitrovic, I. and Napier, T. C., Substance P attenuates and DAMGO potentiates amygdala glutamatergic neurotransmission within the ventral pallidum, Brain Res, 792 (1998) 193-206.
57. Napier, T. C., Simson, P. E., and Givens, B. S., Dopamine electrophysiology of ventral pallidal/substantia innominata neurons: Comparison with the dorsal globus pallidus, J. Pharmacol. Exp. Ther., 258 (1991) 249-262.
58. Tumer, M. S., Lavin, A., Grace, A. A., and Napier, T. C., Regulation of limbic information outflow by the subthalamic nucleus: excitatory amino acid projections to the ventral pallidum, J Neurosci, 21 (2001) 2820-2832.

The embodiments of the invention in which an exclusive property or privilege is claimed are defined as follows:

1. A method of combating methamphetamine addiction or prevention of relapse in a patient experiencing or susceptible to same, by administering to the patient a composition comprising an effective amount of mirtazapine.

2. The method of claim 1, wherein mirtazapine is administered orally to the patient.

3. The method of claim 2, wherein mirtazapine is administered orally in a dose of from about 10 to about 100 milligrams per day.

4. The method of claim 2, wherein mirtazapine is administered orally in a dose of from about 15 to about 60 milligrams per day.

5. A method of combating methamphetamine addiction or prevention of relapse in a patient experiencing or susceptible to same, by administering to the patient a composition comprising an effective amount of 4,5,7a,8,9,10,11,11a,-octahydro-7H-10-methylindolo[1,7,bc][2,6]-napthyridine (SDZ SER 082).

6. The method of claim 5, wherein SDZ SER 082 is administered orally to the patient.

7. The method of claim 6, wherein SDZ SER 082 is administered orally in a dose of from about 10 to about 100 milligrams per day.

8. The method of claim 7, wherein SDZ SER 082 is administered orally in a dose of from about 1 to about 100 milligrams per day.

9. A method of combating methamphetamine addiction or prevention of relapse in a patient experiencing or susceptible to same, by administering to the patient a composition comprising an effective amount of a serotonin antagonist selected from the group consisting of 5-HT_{2A/2C} receptor antagonists and selective 5-HT_{2C} receptor antagonists, wherein the serotonin antagonist has been screened to determine that it does not potentiate the effect of the drug.

10. A method of combating drug addiction or prevention of relapse in a patient experiencing or susceptible to same, by administering to the patient a composition comprising an effective amount of mirtazapine.

11. The method of claim 10, wherein the drug addiction comprises an addictive condition involving one or more of the following drugs: methamphetamine, amphetamine, methylenedioxymethamphetamine (MDMA or ecstasy), and other substituted amphetamines, cocaine, alcohol (ethanol), opiates, and nicotine and other substituted amphetamines.

12. The method of claim 10, wherein said drug addiction is cocaine.

13. The method of claim 10, wherein said drug addiction is heroin.

14. The method of claim 10, wherein said drug addiction is opiates.

15. The method of claim 10, wherein said drug addiction is nicotine.

16. The method of claim 10, wherein said drug addiction is alcohol (ethanol).

17. The method of claim 10, wherein said drug addiction is amphetamine, methylenedioxymethamphetamine (MDMA or ecstasy), and other substituted amphetamines.

18. A method of combating drug addiction or prevention of relapse in a patient experiencing or susceptible to same, by administering to the patient a composition comprising an effective amount of 4,5,7a,8,9,10,11,11a,-octahydro-7H-10-methylindolo[1,7,bc][2,6]-napthyridine (SDZ SER 082).

19. The method of claim 18, wherein the drug addiction comprises an addictive condition involving one or more of the following drugs: methamphetamine, amphetamine, methylenedioxymethamphetamine (MDMA or ecstasy), and other substituted amphetamines, cocaine, alcohol (ethanol), opiates, and nicotine and other substituted amphetamines.

20. The method of claim 18, wherein said drug addiction is cocaine.

21. The method of claim 18, wherein said drug addiction is heroin.

22. The method of claim 18, wherein said drug addiction is opiates.

23. The method of claim 18, wherein said drug addiction is nicotine.

24. The method of claim 18, wherein said drug addiction is alcohol (ethanol).

25. The method of claim 18, wherein said drug addiction is amphetamine, methylenedioxymethamphetamine (MDMA or ecstasy), and other substituted amphetamines.

26. A method for identifying compounds for the treatment of METH addiction, said method comprising:
reversal of behavioral sensitization and/or conditioned place preference in a METH-treated animal in the presence of a known amount of a compound; and
reversal of the electrophysiological endpoints in a METH-treated animal in the presence of a known amount of the compound.

27. The method of claim 26, wherein the compound comprises a 5-HT antagonist.

28. The method of claim 26, wherein the compound comprises a 5-HT_{2A/2C} antagonist or a selective 5-HT_{2C} antagonist.

29. A method for identifying compounds for the treatment of METH addiction, said method comprising:
reversal of behavioral sensitization and/or conditioned place preference in a METH-treated animal in the presence of a known amount of a compound; and
modification of biochemical endpoints in a METH-treated animal in the presence of a known amount of the compound.

30. The method of claim 29, wherein the reversal of behavioral sensitization comprises an attenuation of up-regulated 5-HT_{2A/2C} receptor function or 5-HT_{2C} receptor function in the brain and an attenuation in METH-induced changes in gene transcriptional modulators such as cAMP-response element binding protein.

31. The method of claim 29, further comprising the reversal of the electrophysiological endpoints in a METH-treated animal in the presence of a known amount of the compound.

32. The method of claim 29, wherein the compound comprises a 5-HT antagonist.

33. The method of claim 29, wherein the compound comprises a 5-HT_{2A/2C} antagonist or a selective 5-HT_{2C} antagonist.

34. A method for identifying compounds for the treatment of drug addiction, said method comprising:
reversal of behavioral sensitization and/or conditioned place preference in a drug-treated animal in the presence of a known amount of a compound; and
reversal of the electrophysiological endpoints in a drug-treated animal in the presence of a known amount of the compound.

35. The method of claim 34, wherein the compound comprises a 5-HT antagonist.

36. The method of claim 34, wherein the compound comprises a 5-HT_{2A/2C} antagonist or a selective 5-HT_{2C} antagonist.

37. The method of claim 34, wherein said drug addiction comprises an addictive condition involving one or more of the following drugs: methamphetamine, amphetamine, methylenedioxymethamphetamine (MDMA or ecstasy), and other substituted amphetamines, cocaine, alcohol (ethanol), opiates, and nicotine and other substituted amphetamines.

38. A method for identifying compounds for the treatment of drug addiction, said method comprising:
reversal of behavioral sensitization and/or conditioned place preference in a drug-treated animal in the presence of a known amount of a compound; and
modification of biochemical endpoints in a drug-treated animal in the presence of a known amount of the compound.

39. The method of claim 38, further comprising the reversal of the electrophysiological endpoints in a drug-treated animal in the presence of a known amount of the compound.

40. The method of claim 38, wherein the compound comprises a 5-HT antagonist.

41. The method of claim 38, wherein the compound comprises a 5-HT_{2A/2C} antagonist or a selective 5-HT_{2C} antagonist.

42. The method of claim 38, wherein said drug addiction comprises an addictive condition involving one or more of the following drugs: methamphetamine, amphetamine, methylenedioxymethamphetamine (MDMA or ecstasy), and other substituted amphetamines, cocaine, alcohol (ethanol), opiates, and nicotine and other substituted amphetamines.
